(19) United States
(12) Patent Application Publication
(10) Pub. No.: US 2011/0212181 A1
(43) Pub. Date: Sep. 1, 2011

(54) COMPOSITIONS AND METHODS FOR TREATING CHRONIC RESPIRATORY INFLAMMATION

(75) Inventors: Daisy Kwok Yan Shum, Hong Kong (KG); Mary Sau Man Ip, Hong Kong (HK); Chi Hang Chan, Hong Kong (HK); Valeria On Yue Leung, Hong Kong (HK)

(73) Assignee: The University of Hong Kong, Hong Kong (HK)

(21) Appl. No.: 13/035,414

(22) Filed: Feb. 25, 2011

Related U.S. Application Data
(60) Provisional application No. 61/308,597, filed on Feb. 26, 2010.

(51) Int. Cl. A61K 9/14; A61P 11/00; C08B 37/10; A61K 31/727

(52) U.S. Cl. 424/493; 536/21; 514/56; 424/499

(57) ABSTRACT

Neutrophil elastase (NE) is a protease secreted by neutrophils during inflammation. Aberrant expression of NE such as in chronic respiratory inflammatory diseases, results in tissue destruction and decline in lung function. Compositions including an NE-targeting agent that targets the pathologic elements of respiratory inflammation are provided. Non-anticoagulant heparin derivatives or fragments are exemplary NE-targeting agents. The compositions preferably include a carrier, such as chitosan, to facilitate delivery of the active agent. Methods of manufacturing non-anticoagulant heparin are also provided. Methods of administering the disclosed compositions to treat respiratory diseases are also disclosed. In preferred methods, an effective amount of the pharmaceutical composition is administered to subject in need thereof to reduce, inhibit, or alleviate one or more symptoms of chronic respiratory inflammation. In the most preferred embodiment, the composition is administered as a dry powder, intranasally or by inhalation.

### FIELD OF THE INVENTION

The present application is generally related to compositions that target unopposed activity of neutrophil elastase in recurrent airway inflammation and methods of their use for treatment of such ailments in respiratory diseases.

### RELATED APPLICATIONS

This application claims priority to U.S. Ser. No. 61/308,597 filed Feb. 26, 2010.

### BACKGROUND OF THE INVENTION

Chronic respiratory inflammation (CPI) is a common disease worldwide and poses a heavy economic burden. Poorly-controlled inflammation is the underlying cause of tissue destruction and lung function decline in many respiratory disorders including bronchiectasis and chronic obstructive pulmonary disease (COPD). COPD is a progressive inflammatory disorder characterized by reduced elasticity in the airways and air sacs, and inflammation and deterioration of the walls between the air sacs. This leads to less air flow into and out of the lungs and a variety of symptoms including increased mucus formation, wheezing, shortness of breath, and chest tightness. While tobacco smoking is recognized as a major risk factor, only about 10% of smokers develop chronic respiratory inflammation, suggesting that exposure to other pollutants, genetic factors and childhood infection history may also contribute to the development of the disease. The World Health Organization predicts that COPD will rise from the fifth most common cause of death worldwide to the fourth most common by 2030. This increase is expected to be especially prominent in developing countries.

Currently, treatment of chronic respiratory inflammation is primarily to control symptoms, but has little effect in halting the progression of the disease. Antibiotics are routinely prescribed to control bacterial colonization, and bronchodilators are given to relieve airflow limitation. While these interventions help to control exacerbations and improve symptoms, they do not significantly affect the underlying pathogenic mechanism, and so do not halt the decline of lung function and other deterioration associated with disease progression. Other therapeutic options include mucolytics, anti-inflammatory agents and bronchopulmonary hygiene therapy, however, benefits of these treatments are unclear, and clinical studies of their effects are conflicting. There is, therefore, an urgent need for a drug that targets the pathogenic elements for effective treatment of the chronic respiratory inflammation.

It is therefore an object of the invention to provide methods and compositions that reduce, treat, inhibit, or alleviate one or more symptoms of a respiratory disorder, such as chronic respiratory inflammation.

### SUMMARY OF THE INVENTION

Compositions including a neutrophil elastase (NE)-targeting agent that counters the pathologic elements of respiratory inflammation are provided. NE-targeting agents primarily disrupt the association between NE and shed ectodomains of Syn-1, resulting in inhibition of the NE by protease inhibitors or anti-elastases. Non-anticoagulant heparin derivatives or fragments are exemplary NE-targeting agents. The compositions preferably include a carrier, such as chitosan or lactose in dry powder formulations, to facilitate delivery of the active agent. Methods of manufacturing non-anticoagulant heparin are also provided.

Methods of administering the disclosed compositions to treat respiratory diseases and disorders are also disclosed. Methods include administering an effective amount of a pharmaceutical composition including an NE-targeting agent to a subject in need thereof to reduce neutrophil elastase activity. In preferred methods, an effective amount of the pharmaceutical composition is administered to a subject in need thereof to reduce, inhibit, or alleviate one or more symptoms of chronic respiratory inflammation. In the most preferred embodiment, the composition is administered as a dry powder, intranasally or by inhalation. Kits including NE-targeting agents are also provided.

### BRIEF DESCRIPTION OF THE DRAWINGS

FIG. 1 is a graph illustrating the elution profile of heparin saccharides fragmented by heparitinase-III and separated by gel filtration. Sample absorbance at 232 nm (-♦-) and cetylpyridinium chloride (CPC) turbidity test (-═-) are plotted as a function of fraction number. Peaks I-IV represent octa, hexa, tetra, and di heparin saccharides respectively. V₀=void volume, Vₜ=total volume.

FIG. 2 A is a graph illustrating the inhibition of Factor Xa activity (absorbance) by commercial undigested heparin (porcine intestinal product) (-♦-), and the octa- (-▪-), hexa- (-▴-), tetra- (-∘-), and di- (-•-) saccharide fragments as a function of concentration (μM). FIG. 2 B is a graph illustrating the inhibition of Factor IIa activity (absorbance) by commercial undigested heparin (-♦-), and the octa- (-▪-), hexa- (-▴-), tetra- (-∘-), and di- (-•-) saccharide fragments as a function of concentration (μM).

FIG. 3 is a bar graph illustrating the number of neutophils (as a percentage of total cells found in bronchoalveolar lavage fluid (BALF)) in sham air group (white bars) administered chitosan (left-hand, white bar) or Hp-chitosan (right-hand, white bar), and cigarette smoking group (black bars) administered chitosan (left-hand, black bar) or Hp-chitosan (right-hand, black bar). *=P less than 0.001.

FIG. 4 is a bar graph illustrating the concentration of neutrophil elastase (NE) (nM) in sham air group with chitosan administration (vertical hatching), sham air group with Hp-chitosan administration (horizontal hatching), smoking group with chitosan administration (solid black), and smoking group with Hp-chitosan administration (diagonal hatching). *=P less than 0.001.

FIG. 5 is a bar graph illustrating the molar ratio of alpha-1-antitrypsin (α₁AT) in relation to neutrophil elastase (NE) in sham air group (white bars) administered chitosan (left-hand, white bar) or Hp-chitosan (right-hand, white bar), and cigarette smoking group (black bars) administered chitosan (left-hand, black bar) or Hp-chitosan (right-hand, black bar). **=P less than 0.01.

FIG. 6 is a bar graph illustrating the myeloperoxidase (MPO) activity (mU/ml) in sham air group (white bars) administered chitosan (left-hand, white bar) or Hp-chitosan (right-hand, white bar), and cigarette smoking group (black bars) administered chitosan (left-hand, black bar) or Hp-chitosan (right-hand, black bar). These units measure the activity of MPO in the bronchoalveolar lavage fluid of the rats. One unit of MPO activity was defined as that degrading 1 μmole of peroxide per minute at 37° C. *=P less than 0.001.

FIG. 7 is a bar graph illustrating the airspace enlargement as a measure of mean linear intercept (Lm (μm)) in sham air group (white bars) administered chitosan (left-hand, white bar) or Hp-chitosan (right-hand, white bar), and cigarette smoking group (black bars) administered chitosan (left-hand, black bar) or Hp-chitosan (right-hand, black bar). *=P less than 0.001. **=P less than 0.01.

### DETAILED DESCRIPTION OF THE INVENTION

### I. Definitions

The term “effective amount” or “therapeutically effective amount” with regard to the disclosed diseases means a dosage sufficient to reduce, prevent, or inhibit one or more biochemical measures or symptoms associated with a respiratory disease or disorder such as chronic pulmonary inflammation, or otherwise provide a desired pharmacologic and/or physiologic effect. These terms can also be used with regard to a reduction in neutrophil elastase (NE) binding to syndecan-1, or a reduction in NE activity. The precise dosage will vary according to a variety of factors such as subject-dependent variables (e.g., age, immune system health, etc.), the disease, and the treatment being effected.

The terms “individual,” “subject,” and “patient” are used interchangeably herein, and refer to a mammal, including, but not limited to, rodents, simians, humans, mammalian farm animals, mammalian sport animals, and mammalian pets.

The term “heparin derivative” as used herein includes, but is not limited to, substances resulting from modifications along the linear chain of native heparin or fragments thereof, and fragments of native heparin.

The term “syndecan-1 (Syn-1) as used herein refers collectively to syndecan-1 and shed ectodomains of syndecan-1.

### II. Compositions

A. Neutrophil elastase targeting Agents

Neutrophil elastase (NE) (also referred to as leukocyte elastase, and

ELA2 (elastase 2, neutrophil)) is a serine protease with broad substrate specificity. Secreted by neutrophils during airway infection/inflammation, NE destroys bacteria and, when unopposed, host tissue. However, when expressed aberrantly as in chronic respiratory inflammatory diseases including bronchiectasis and chronic obstructive pulmonary disease (COPD), persistent NE activity causes extensive tissue damage and recurrent inflammation. In the inflamed airways, NE is found in association with shed syndecan-1 (Syn-1), a cell surface heparan sulfate proteoglycan released into the airways as a result of chronic inflammation (Chan, et al., Am. J. Respir. Cell Mol. Biol., 41(5):620-8 (2009)). This association prevents the action of endogenous NE inhibitors. NE therefore remains active, digesting structural components of the airways and resulting in lung injury.

In healthy individuals, the activity of neutrophil elastase (NE) is effectively controlled by α₁-antitrypsin. Unregulated NE causes extensive tissue damage and recurrent inflammation. Compositions for inhibiting or reducing NE activity containing an NE-targeting agent are provided. An NE-targeting agent can inhibit the activity of NE directly, such as by blocking the active site of the enzyme. Alternatively, an NE-targeting agent can disrupt the association between NE and syndecan-1, or shed ectodomains thereof. Disruption of the association between NE and Syn-1 allows protease inhibitors or anti-elastases such as alpha 1-antitrypsin (α₁-antitrypsin or α₁AT) to inhibit the activity of NE. Representative NE-targeting agents include, but are not limited to, glycosaminoglycans, peptides, antibodies, or small molecules. In a preferred embodiment, the NE-targeting agent is a heparin, or a derivative, analog, or fragment, thereof. In a more preferred embodiment, the heparin, heparin derivative, or fragment thereof exhibits reduced anti-coagulation activity. In the most preferred embodiment, the NE-targeting agent is a non-anticoagulant heparin derivative that disrupts the association between NE and Syn-1.

In certain embodiments, the NE-targeting agent is a small molecule, for example, a molecule of about 500 Daltons. The small molecules can be obtained by screening a library of compounds for binding to, and reducing the activity of NE bound to Syn-1. Such screening techniques are routine and known in the art.

Native heparin is a polymer with a molecular weight ranging from 3 kDa to 50 kDa, although the average molecular weight of most commercial heparin preparations is in the range of 12 kDa to 15 kDa. Heparin is a member of the glycosaminoglycan family of carbohydrates (which includes the closely-related molecule heparan sulfate) and consists mainly of domains of highly-sulfated repeating (hexuronate-hexosamine) disaccharide units. The amino sugar is more frequently N-sulfated glucosamine (GlcNS) than N-acetylglucosamine (GlcNAc), and the hexuronic acid is more frequently iduronic acid (IdoA) than glucuronic acid (GlcA). Other modifications may be found along the linear chain of heparin. The glucosamine residues may be N-sulfated and possibly N-unsubstituted. In addition, O-sulfation may be found at C2 of IdoA and C6 of glucosamine, and possibly at C3 of GlcNS and C2 of GlcA.

The anti-coagulant activity of heparin is attributed to the pentasaccharide sequence: GlcNAc,6S- GlcA- GlcNS,3S,6S- IdoA,2S- GlcNS,6S. This pentasaccharide sequence binds and activates antithrombin (AT), which in turn inhibits the coagulation cascade and prevents blood clotting.

In preferred embodiments, the heparin derivative has reduced anticoagulant activity compared to native heparin, while maintaining its ability to function as a NE-targeting agent. The anticoagulant activity of the heparin, heparin derivative, or fragment thereof, is at least 80%, more preferably 90%, still more preferably 95% lower, when compared to therapeutic anticoagulant heparin. In the most preferred embodiment, the heparin does not have anticoagulant activity. The use of a non-anticoagulant heparin derivative is preferred to minimize the possibility of hemorrhagic side effects of heparin. As shown in Example 1 below, the anticoagulant activity of therapeutic heparin or its derivatives may be determined by assays such as activated partial thromboplastin time (aPTT), prothrombin time (PT), heparin antifactor Xa assay and heparin antifactor IIa assay.

In a preferred embodiment, therapeutic heparin, heparin derivatives, or fragments thereof have an average molecular weight of from 1 to 10 kD, more preferably from 1.5 to 6 kD, still more preferably from 2 to 5 kD. Heparin fragments can be 2 to 4 disaccharide units (tetra- to octasaccharides). Preferred fragments are described in Example 1 below. In a preferred embodiment, the fragments are tetra-, hexa- or octasaccharides of heparin, or a heparin derivative. In another embodiment, a fragment of heparin or a heparin derivative is defined by the number of glucuronic acid (GlcA) residues in the fragment. In one embodiment the number of GlcA residues is between 1 and 10, preferably between 1 and 5. The compositions may be composed of fragments with uniform length or a mixture of chains with various lengths. The composition may therefore contain therapeutic heparin, heparin derivatives or fragments, of uniform sequence or of a mixture of sequences.

In the most preferred embodiments the NE-targeting agent is one or more fragments of non-anticoagulant heparin that can disrupt or dissociate the interaction between NE and Syn-1. Disrupting this association allows endogenous or exogenous protease inhibitors or anti-elastases to decrease the activity of NE. Methods for determining if a heparin derivative or fragment can disrupt or dissociate the association between NE and Syn-1 are known in the art, and include competition assays such as surface plasmon resonance. See for example Chan, et al., Am. J. Respir. Cell Mol. Biol., 41(5):620-8 (2009).

Heparin, heparin derivatives and fragments thereof may be derived from any suitable commercially available heparin or heparin salt. The parent heparin may or may not be fractionated. The parent heparin may be isolated from a natural source, e.g. from an animal. Heparin may be obtained from animals such as pigs, cattle, sharks and squids, in the tissues such as skin, lung, intestinal mucosa and cartilage that express heparin sulfate proteoglycans. Alternatively, the parent heparin may be synthesized by cells engineered to express heparin sulfate proteoglycans or by chemical synthesis.

Non-anticoagulant heparin derivatives, or fragments may be generated from the parent heparin by fragmentation or depolymerization. Fragmentation or depolymerization may be achieved using enzymes such as heparitinase I, heparitinase II and heparitinase III, or chemicals such as nitrous acid. Digestion by heparitinase III is particularly preferred, as the enzyme selectively cleaves highly sulfated regions of the heparin chain, where the antithrombin-binding pentasaccharide sequence resides. Thus, the resultant digestion product will unlikely bind antithrombin and will not possess anticoagulant activity. Non-anticoagulant heparin may also be generated by chemical modification, for example acetylation, O-desulfation and N-desulfation. Alternatively, non-anticoagulant heparin, heparin derivatives, or fragments thereof may be produced by chemical synthesis.

B. Formulations

The disclosed NE-targeting agents can be administered solely as active agent or more preferably in combination with a suitable pharmaceutical carrier for the mode of administration. The term “pharmaceutically acceptable” means a non-toxic material that does not interfere with the effectiveness of the biological activity of the active ingredients. The term “pharmaceutically-acceptable carrier” means one or more compatible solid or liquid fillers, dilutants or encapsulating substances for administration to a human or other vertebrate animal. The term “carrier” refers to an organic or inorganic ingredient, natural or synthetic, with which the active ingredient is combined to facilitate the application. Pharmaceutically acceptable carriers are determined in part by the particular composition being administered, as well as by the particular method used to administer the composition.

Pharmaceutical compositions may be formulated in a conventional manner using one or more physiologically acceptable carriers including excipients and auxiliaries which facilitate processing of the active compounds into preparations which can be used pharmaceutically. The compositions may be administered in combination with one or more physiologically or pharmaceutically acceptable carriers, salts, buffering agents, emulsifiers, diluents, excipients, chelating agents, fillers, drying agents, antioxidants, antimicrobials, preservatives, binding agents, bulking agents, silicas, solubilizers, stabilizers, surfactants, thickening agents, co-solvents, adhesives, viscosity and absorption enhancing agents and agents capable of adjusting osmolarity of the formulation. Proper formulation is dependent upon the route of administration chosen. If desired, the compositions may also contain minor amount of nontoxic auxiliary substances such as wetting or emulsifying agents, dyes, pH buffering agents, or preservatives. Accordingly, there is a wide variety of suitable formulations of pharmaceutical compositions containing the NE-targeting agent some of which are described herein.

In preferred embodiments, a carrier is employed to facilitate delivery of the disclosed NE-targeting agents. For example, heparin, heparin derivatives, and fragments thereof are polyanionic and therefore likely to form aggregates. A carrier, excipient or other materials are therefore used to improve effective delivery by inhalation. Preferred carriers are biodegradable, non-toxic, and do not affect the activity of the NE-targeting agent. The carrier may take any form, for example liposomes, scaffolds, micelles, capsules, beads, spheres or droplets. The carrier may be made of one, or a combination of materials, such as a carbohydrate, glycoprotein, polyamino acid, or biocompatible biodegradable polymer, such as chitosan, poly(L-lysine), poly(ethylene glycol) and polylactide-glycolide. The carrier is loaded with the NE-targeting agent before administration, and upon reaching the target site will release the NE-targeting agent.

In preferred carriers, release is controlled by the change in pH from pH 4-5.5, more preferably pH 4.5-5.0, and still more preferably pH 4.7-4.9, to a higher pH of pH 7-8.5, more preferably pH 7.2-8.2, and still more preferably pH 7.5-8.0. The release of NE-targeting agent may occur in 1 to 48 hr, more preferably in 6 to 36 hr, and even more preferably in 12 to 24 hr. The carrier will ultimately be degraded and eliminated by the body.

In a preferred embodiment, the carrier is chitosan or a chitosan derivative. Chitosan is a linear polysaccharide composed of randomly distributed β-(1-4)-linked D-glucosamine (deacetylated unit) and N-acetyl-D-glucosamine (acetylated unit). It is a preferred vehicle for NE-targeting agent delivery because it is biocompatible, biodegradable, bioadhesive, and exhibits favorable physiochemical properties (Lee, et al., Respir. Res., 7(112):1-10 (2006)). Methods for producing chitosan particles by membrane emulsification are described in Wang, et al., J. Control. Release, 106(1-2):62-75 (2005). Microspheres, or beads of uniform size can be prepared by utilizing membranes that differ in pore size. As described in U.S. Patent Application 20080202513, chitosan can be formulated for dry powder delivery by inhalation. Particulate materials having an average diameter within the range 0.5 μm to 11 μm are an appropriate size to enter the lungs. Particles having an average diameter of less than 0.5 μm are generally so small that if inhaled, they may not be trapped within the lung but may be exhaled. Particles having an average diameter of more than 13 μm are generally too large to enter the upper airways of the lungs. A fraction of dry powder composition comprising particles having an average diameter within the range 0.5 to 11 μm is thus deemed a respirable fraction. Microspheres and beads preferably range in size from an average diameter of 2 to 10 μm, more preferably 4 to 8 μm, and even more preferably 4.5 to 5.5 μm. In the most preferred embodiment the carrier will have a hydrodynamic diameter of 5 μm, which targets the medication to the lower respiratory tract. The size of the carrier may be chosen to deliver the NE-targeting agent to any desired location, from the upper respiratory tract to the lungs.

Pharmaceutical compositions including effective amounts of NE-targeting active agent suitable for intranasal or pulmonary delivery are disclosed. In a preferred embodiment, formulations are for dry powder. Substances may be included, for example, to dilute the powder to an amount which is suitable for delivery from the particular intended powder inhaler; to facilitate the processing of the preparation; to improve the powder properties of the preparation; to improve the stability of the preparation, e.g. by means of antioxidant or pH-adjusting compounds; or to add a taste to the preparation. Any additive should not adversely affect the stability of the NE-targeting agent, or disadvantageously interfere with absorption of the NE-targeting agent. It should also be stable, not hygroscopic, have good powder properties and have no adverse effects in the airways. Examples of additives include, but are not limited to mono-, di-, and polysaccharides, sugar alcohols and other polyols, such as for example lactose, glucose, raffinose, melezitose, lactitol, maltitol, trehalose, sucrose, mannitol and starch. Depending upon the inhaler to be used, the total amount of such additives may vary over a very wide range.

Pharmaceutical compositions may be formulated in a conventional manner using one or more physiologically acceptable carriers including excipients and auxiliaries which facilitate processing of the active compounds into preparations which can be used pharmaceutically. The compositions may be administered in combination with one or more physiologically or pharmaceutically acceptable carriers, salts, buffering agents, emulsifiers, diluents, excipients, chelating agents, fillers, drying agents, antioxidants, antimicrobials, preservatives, binding agents, bulking agents, silicas, solubilizers, stabilizers, surfactants, thickening agents, co-solvents, adhesives, viscosity and absorption enhancing agents and agents capable of adjusting osmolarity of the formulation. Proper formulation is dependent upon the route of administration chosen. If desired, the compositions may also contain minor amount of nontoxic auxiliary substances such as wetting or emulsifying agents, dyes, pH buffering agents, or preservatives.

In preferred embodiments, a carrier is employed to facilitate delivery of NE-targeting agent. For example, heparin, heparin derivatives, and fragments thereof are highly anionic and therefore likely to form aggregates. A carrier, excipient or other materials is used to improve effective delivery by inhalation. Preferred carriers are biodegradable, non-toxic, and do not affect the activity of the NE-targeting active agent. The carrier may take any form, for example, liposomes, scaffolds, micelles, capsules, beads, spheres or droplets. The carrier may be made of one, or a combination of materials, such as a carbohydrate, glycoprotein, polyamino acid, or biocompatible biodegradable polymer, such as chitosan, poly(L-lysine), poly(ethylene glycol) and polylactide-glycolide. The carrier is loaded with the NE-targeting agent before administration, and upon reaching the target site will release the NE-targeting agent.

Dry powder formulations (DPF's) are gaining increased interest as aerosol formulations for pulmonary delivery. Dry powder aerosols for inhalation therapy are generally produced with mean geometric diameters primarily in the range of less than 5μ. Dry powder formulations with large particle size have been shown to possess improved flowability characteristics, such as less aggregation, easier aerosolization, and potentially less phagocytosis. Inhalation devices which can be employed to deliver dry powder formulations to the lungs include non-breath-activated or “multistep” devices. In these devices, the drug formulation is first dispersed by energy independent of a patient's breath, then inhaled. Other examples of inhalers include the SPINHALER® (Fisons, Loughborough, U.K.) and ROTAHALER® (Glaxo-Wellcome, Research Triangle Park, N.C.). Nebulizers, such as described by Cipolla et al. (Cipolla et al. Respiratory Drug Delivery VII, Biological, Pharmaceutical, Clinical and Regulatory Issues Relating to Optimized Drug Delivery by Aerosol, Conference held May 14-18, 2000, Palm Springs, Fla., also are employed in pulmonary delivery.

The particles can include excipients such as buffer salts, dextran, polysaccharides, lactose, trehalose, cyclodextrins, proteins, polycationic complexing agents, peptides, polypeptides, fatty acids, fatty acid esters, inorganic compounds, phosphates, lipids, sphingolipids, cholesterol, surfactants, polyaminoacids, polysaccharides, proteins, salts, gelatins, and polyvinylpyrridolone.

As used herein, the term “surfactant” refers to any agent which preferentially adsorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to microparticles, they tend to present moieties to the external environment that do not attract similarly-coated particles, thus reducing particle aggregation. Surfactants may also promote absorption of a therapeutic or diagnostic agent and increase bioavailability of the agent. Suitable surfactants include but are not limited to hexadecanol; fatty alcohols such as polyethylene glycol (PEG); polyoxyethylene-9-laury-1 ether; a surface active fatty acid, such as palmitic acid or oleic acid; glycocholate; surfactin; a poloxamer; a sorbitan fatty acid ester such as sorbitan trioleate (Span 85); Tween 80 and tyloxapol. Phospholipids suitable for delivery to a human include phosphatidylcholines dipalmitoyl phosphatidylcholine (DPPC), dipalmitoyl phosphatidylethanolamine (DPPE), distearoyl phosphatidylcholine (DSPC), dipalmitoyl phosphatidyl glycerol (DPPG) or any combination thereof. Methods of preparing and administering particles including surfactants, and in particular phospholipids, are disclosed in U.S. Pat. No. RE 37,053 to Hanes et al. and U.S. Pat. No. 5,985,309.

Particles which have a tap density of less than about 0.4 g/cm³ are referred herein as “aerodynamically light particles”. More preferred are particles having a tap density less than about 0.3 g/cm³, less than about 0.2 g/cm³, most preferably less than about 0.1 g/cm³. Tap density can be determined using the method of USP Bulk Density and Tapped Density, United States Pharmacopeia convention, Rockville, Md., 10th Supplement, 4950-4951, 1999. Instruments for measuring tap density known to those skilled in the art include the Dual Platform Microprocessor Controlled Tap Density Tester (Vankel, N.C.) or a GeoPyc instrument (Micrometrics Instrument Corp., Norcross, Ga. 30093). Tap density is a standard measure of the envelope mass density. The envelope mass density of an isotropic particle is defined as the mass of the particle divided by the minimum spherical envelope volume within which it can be enclosed. Features which can contribute to low tap density include irregular surface texture and porous structure.

Aerodynamically light particles have a preferred size, e.g., a volume median geometric diameter (VMGD) greater than about 5 microns. In one embodiment, the VMGD is from greater than about 5μ to about 30μ. In other embodiments, the particles have a median diameter, mass median diameter (MMD), a mass median envelope diameter (MMED) or a mass median geometric diameter (MMGD) greater than about 5μ, for example from greater than about 5μ and about 30μ. The diameter of the spray-dried particles, for example, the VMGD, can be measured using a laser diffraction instrument (for example Helos, manufactured by Sympatec, Princeton, N.J.). Other instruments for measuring particle diameter are well known in the art. The diameter of particles in a sample will range depending upon factors such as particle composition and methods of synthesis. The distribution of size of particles in a sample can be selected to permit optimal deposition to targeted sites within the respiratory tract.

In preferred carriers, release is controlled by the change in pH from pH 4-5.5, more preferably pH 4.5-5.0, and still more preferably pH 4.7-4.9, to a higher pH of pH 7-8.5, more preferably pH 7.2-8.2, and still more preferably pH 7.5-8.0. The release of NE-targeting agent may occur in 1 to 48 hr, more preferably in 6 to 36 hr, and even more preferably in 12 to 24 hr. The carrier will ultimately be degraded and eliminated by the body.

In a preferred embodiment, the carrier is chitosan or a chitosan derivative. Chitosan is a linear polysaccharide composed of randomly distributed β-(1-4)-linked D-glucosamine (deacetylated unit) and N-acetyl-D-glucosamine (acetylated unit). It is a preferred vehicle for NE-targeting agent delivery because it is biocompatible, biodegradable, bioadhesive, and exhibits favorable physiochemical properties (Lee, et al., Respri. Res., 7(112):1-10 (2006)). Methods for producing chitosan particles by membrane emulsification are described in Wang, et al., J. Control Release, 106(1-2):62-75 (2005). Microspheres or beads of uniform size can be prepared by utilizing membranes of different pore size. As described in U.S. Patent Application 20080202513, chitosan can be formulated for dry powder delivery by inhalation. Particulate materials having an average diameter within the range 0.5 μm to 11 μm are an appropriate size to enter the lungs. Particles having an average diameter of less than 0.05 μm are generally so small that if inhaled, they may not be trapped within the lung but may be exhaled. Particles having an average diameter of more than 13 μm are generally too large to enter the upper airways of the lungs. A fraction of dry powder composition comprising particles having an average diameter within the range 0.5 to 11 μm is deemed a respirable fraction. Microspheres and beads preferably range in size from an average diameter of 2 to 10 μm, more preferably 4 to 8 μm, and even more preferably 4.5 to 5.5 μm. In the most preferred embodiment the carrier will have a diameter of 5 μm, which targets the medication to the lower respiratory tract. The size of the carrier may be chosen to deliver the NE-targeting agent to any desired location, from the upper respiratory tract to the lungs.

For administration via the upper respiratory tract, the composition can also be formulated into a solution, e.g., water or isotonic saline, buffered or unbuffered, or as a suspension, at an appropriate concentration for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's Pharmaceutical Sciences 16th edition, Ed. Arthur Osol, page 1445 (1980)). One skilled in the art can readily determine a suitable saline content and pH for an innocuous aqueous solution for nasal and/or upper respiratory administration.

Mucosal formulations may optionally include one or more agents for enhancing delivery through the nasal mucosa. Agents for enhancing mucosal delivery are known in the art, see for example U.S. Patent Application No. 20090252672, to Eddington, and U.S. Patent Application No. 20090047234, to Touitou. Agents include, but are not limited to, chelators of calcium (EDTA), inhibitors of nasal enzymes (boro-leucin, aprotinin), inhibitors of muco-ciliary clearance (preservatives), solubilizers of nasal membrane (cyclodextrin, fatty acids, surfactants) and formation of micelles (surfactants such as bile acids, Laureth 9 and taurodehydrofusidate (STDHF)).

The composition alone or in combination with other suitable components, can be “nebulized” to be administered via inhalation. Aerosol formulations can be placed into pressurized acceptable propellants. For administration by inhalation, the compounds are conveniently delivered in the form of an aerosol spray presentation from pressurized packs or a nebulizer, with the use of a suitable propellant.

Drug may be encapsulated by, or formed into a matrix with, one or more polymers. Polymers include both synthetic and natural polymers, either non-biodegradable or biodegradable and either water soluble or water insoluble. Representative synthetic polymers include polyethylene glycol (“PEG”), polyvinyl pyrrolidone, polymethacrylates, polylysine, poloxamers, polyvinyl alcohol, polyacrylic acid, polyethylene oxide, and polyethyoxazoline. Representative natural polymers include albumin, alginate, gelatin, acacia, chitosan, cellulose dextran, ficoll, starch, hydroxyethyl cellulose, hydroxypropyl cellulose, hydroxy-propylmethyl cellulose, hyaluronic acid, carboxyethyl cellulose, carboxymethyl cellulose, deacetylated chitosan, dextran sulfate, and derivatives thereof. Preferred polymers include PEG, polyvinyl pyrrolidone, poloxamers, hydroxypropyl cellulose, and hydroxyethyl cellulose. The polymer is selected for use in a particular drug matrix formulation based on a variety of factors, such as the polymer molecular weight, polymer hydrophilicity, and polymer inherent viscosity. The polymer can be used as a bulking agent, as an anti-crystallization agent for drugs in an amorphous state, as a crystal growth inhibitor for drugs in a crystalline state or as a wetting agent.

The amount of polymer in the drug matrix is less than about 95%, more preferably less than about 80%, by weight of the drug matrix when used as a bulking agent. The amount of polymer in the drug matrix is less than about 50%, more preferably less than about 40%, by weight of the drug matrix when used as an anti-crystallization agent for drugs in an amorphous state or as a crystal growth inhibitor for drugs in a crystalline state. The amount of polymer in the drug matrix is less than about 30%, more preferably less than about 20%, by weight of the drug matrix when used a wetting agent.

Representative sugars that can be used in drug matrices include mannitol, sorbitol, xylitol, glucitol, ducitol, inositiol, arabinitol, arabitol, galactitol, iditol, allitol, fructose, sorbose, glucose, xylose, trehalose, allose, dextrose, altrose, glucose, idose, galactose, talose, ribose, arabinose, xylose, lyxose, sucrose, maltose, lactose, lactulose, fucose, rhamnose, melezitose, maltotriose, and raffinose. The sugars can serve as a bulking agent or as an anti-crystallization agent for drugs in the amorphous state, or as a crystal growth inhibitor for drugs in the crystalline state or to provide wetting of the porous drug matrix or the drug microparticles within the matrix. The amount of sugar in the drug matrix is typically less than about 95%, more preferably less than about 80%, by weight of the drug matrix when used as a bulking agent and less than about 50%, more preferably less than about 40%, by weight of the drug matrix when used as an anti-crystallization agent for drugs in an amorphous state or as a crystal growth inhibitor for drugs in a crystalline state. The amount of sugar in the drug matrix is less than about 30%, more preferably less than about 20%, by weight of the drug matrix when used a wetting agent.

Representative amino acids that can be used in the drug matrices include both naturally occurring and non-naturally occurring amino acids. The amino acids can be hydrophobic or hydrophilic and may be D amino acids, L amino acids or racemic mixtures. Amino acids which can be used include, but are not limited to: glycine, arginine, histidine, threonine, asparagine, aspartic acid, serine, glutamate, proline, cysteine, methionine, valine, leucine, isoleucine, tryptophan, phenylalanine, tyrosine, lysine, alanine, glutamine. The amino acid can be used as a bulking agent, or as an anti-crystallization agent for drugs in the amorphous state, or as a crystal growth inhibitor for drugs in the crystalline state or as a wetting agent. Hydrophobic amino acids such as leucine, isoleucine, alanine, glucine, valine, proline, cysteine, methionine, phenylalanine, tryptophan are more likely to be effective as anticrystallization agents or crystal growth inhibitors. In addition, amino acids can serve to make the matrix have a pH dependency that can be used to influence the pharmaceutical properties of the matrix such as solubility, rate of dissolution or wetting. The amount of amino acid in the drug matrix is less than about 95%, more preferably less than about 80%, by weight of the drug matrix when used as a bulking agent. The amount of amino acid in the drug matrix is less than about 50%, more preferably less than about 40%, by weight of the drug matrix when used as an anti-crystallization agent for drugs in an amorphous state or as a crystal growth inhibitor for drugs in a crystalline state. The amount of amino acid in the drug matrix is less than about 30%, more preferably less than about 20%, by weight of the drug matrix when used a wetting agent.

Preservatives such as parabens or benzoic acids can be used directly for inhibition of microbial growth. Preferred parabens include methyl paraben, ethyl paraben and butyl paraben. In addition, the preservatives can be used to interact with the drug to inhibit crystal formation or growth. The amount of preservative in the drug matrix is less than about 50%, more preferably less than about 40%, by weight of the drug matrix when used as an anti-crystallization agent for drugs in an amorphous state or as a crystal growth inhibitor for drugs in a crystalline state.

Wetting agents can be used to facilitate water ingress into the matrix and wetting of the drug particles in order to facilitate dissolution. Representative examples of wetting agents include gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., TWEEN®), polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxy propylcellulose, hydroxypropylmethylcellulose phthlate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent. Most of these wetting agents are known pharmaceutical excipients and are described in detail in the Handbook of Pharmaceutical Excipients, published jointly by the American Pharmaceutical Association and The Pharmaceutical Society of Great Britain (The Pharmaceutical Press, 1986). Two or more wetting agents can be used in combination. The amount of wetting agent in the drug matrix is less than about 30%, more preferably less than about 20%, by weight of the drug matrix.

Porous drug particles may be formed by dissolving drug and excpient in a volatile solvent and spray drying. The choice of solvent depends on the drug. In a preferred embodiment, the solvent is an organic solvent that is volatile, has a relatively low boiling point, or can be removed under vacuum, and which is acceptable for administration to humans in trace amounts. Representative solvents include acetic acid, acetaldehyde dimethyl acetal, acetone, acetonitrile, chloroform, chlorofluorocarbons, dichloromethane, dipropyl ether, diisopropyl ether, N,N-dimethlyformamide (DMF), foramide, demethyl sulfoxide (DMSO), dioxane, ethanol, ethyl acetate, ethyl formate, ethyl vinyl ether, methyl ethyl ketone (MEK), glycerol, heptane, hexane, isopropanol, methanol, isopropanol, butanol, triethylamine, nitromethane, octane, pentane, tetrahydrofuran (THF), toluene, 1,1,1-trichloroethane, 1,1,2-trichloroethylene, water, xylene, and combinations thereof. In general, the drug is dissolved in the volatile solvent to form a drug solution having a concentration of between 0.01 and 80% weight to volume (w/v), more preferably between 0.025 and 30% (w/v). When the drug is a water-soluble drug, aqueous solvents or mixtures of aqueous and organic solvents, such as water-alcohol mixtures, can be used to dissolve the drug.

Pore forming agents are volatile materials that are used during the process to create porosity in the resultant matrix. The pore forming agent can be a volatilizable solid or volatilizable liquid. Liquid pore forming agent must be immiscible with the drug solvent and volatilizable under processing conditions compatible with the drug. To effect pore formation, the pore forming agent first is emulsified with the drug solvent. Then, the emulsion is further processed to remove the drug solvent and the pore forming agent simultaneously or sequentially using evaporation, vacuum drying, spray drying, fluid bed drying, lyophilization, or a combination of these techniques. The selection of liquid pore forming agents will depend on the drug solvent. Representative liquid pore forming agents include water; dichloromethane; alcohols such as ethanol, methanol, or isopropanol; acetone; ethyl acetate; ethyl formate; dimethylsulfoxide; acetonitrile; toluene; xylene; dimethylforamide; ethers such as THF, diethyl ether, or dioxane; triethylamine; foramide; acetic acid; methyl ethyl ketone; pyridine; hexane; pentane; furan; water; and cyclohexane. Liquid pore forming agent is used in an amount that is between 1 and 50% (v/v), preferably between 5 and 25% (v/v), of the drug solvent emulsion.

Solid pore forming agent must be volatilizable under processing conditions which do not harm the drug compositions. The solid pore forming agent can be (i) dissolved in the drug solution, (ii) dissolved in a solvent which is not miscible with the drug solvent to form a solution which is then emulsified with the drug solution, or (iii) added as solid particulates to the drug solution. The solution, emulsion, or suspension of the pore forming agent in the drug solution then is further processed to remove the drug solvent, the pore forming agent, and, if appropriate, the solvent for the pore forming agent simultaneously or sequentially using evaporation, spray drying, fluid bed drying, lyophilization, vacuum drying, or a combination of these techniques. The solid pore forming agent is a volatile salt, such as salts of volatile bases combined with volatile acids. Volatile salts are materials that can transform from a solid or liquid to a gaseous state using added heat and/or vacuum. Examples of volatile bases include ammonia, methylamine, ethylamine, dimethylamine, diethylamine, methylethylamine, trimethylamine, triethylamine, and pyridine. Examples of volatile acids include carbonic acid, hydrochloric acid, hydrobromic acid, hydroiodic acid, formic acid, acetic acid, propionic acid, butyric acid, and benzoic acid. Preferred volatile salts include ammonium bicarbonate, ammonium acetate, ammonium chloride, ammonium benzoate and mixtures thereof. Other examples of solid pore forming agents include iodine, phenol, benzoic acid (as acid not as salt), and naphthalene. The solid pore forming agent is used in an amount between 5 and 1000% (w/w), preferably between 10 and 600% (w/w), and more preferably between 10 and 200% (w/w), of the drug.

Porous drug matrices preferably are made by (i) dissolving a drug, preferably one having low aqueous solubility, in a volatile solvent to form a drug solution, (ii) combining at least one pore forming agent with the drug solution to form an emulsion, suspension, or second solution, and (iii) removing the volatile solvent and pore forming agent from the emulsion, suspension, or second solution. In a preferred embodiment, spray drying, optionally followed by lyophilization or vacuum drying, is used to remove the solvents and the pore forming agent. The removal of the pore forming agent can be conducted simultaneously with or following removal of enough solvent to solidify the droplets. Production can be carried out using continuous, batch, or semi-continuous processes. First, the selected drug is dissolved in an appropriate solvent. The concentration of the drug in the resulting drug solution typically is between about 0.01 and 80% (w/v), preferably between about 0.025 and 30% (w/v). Next, the drug solution is combined, typically under mixing conditions, with the pore forming agent or solution thereof. If a liquid pore forming agent is used, it is first emulsified with the drug solution to form droplets of pore forming agent dispersed throughout the drug solution. If a solid pore forming agent is used, it is dissolved either directly in the drug solution to form a solution of drug/pore forming agent, or it is first dissolved in a second solvent which is immiscible with the drug solvent to form a solution which subsequently is emulsified with the drug solution to form droplets of the pore forming agent solution dispersed throughout the drug solution. Subsequently, the solid pore forming agent particle size can be reduced by further processing the resulting suspension, for example, using homogenization or sonication techniques known in the art. In the preferred embodiment, excipient(s) are added to the emulsion, suspension or second solution before, with or after the pore-forming agent. The solution, emulsion, or suspension is further processed to remove the drug solvent and the pore forming agent simultaneously or sequentially, using evaporation, spray drying, fluid bed drying, lyophilization, vacuum drying, or a combination of these techniques. In a preferred embodiment, the solution, emulsion, or suspension is spray-dried. As used herein, “spray dry” means to atomize the solution, emulsion, or suspension to form a fine mist of droplets (of drug solution having solid or liquid pore forming agent dispersed throughout), which immediately enter a drying chamber (e.g., a vessel, tank, tubing, or coil) where they contact a drying gas. The solvent and pore forming agents evaporate from the droplets into the drying gas to solidify the droplets, simultaneously forming pores throughout the solid. The solid (typically in a powder, particulate form) then is separated from the drying gas and collected.

In embodiments in which at least one pore forming agent is combined with the drug solution to form an emulsion, a surfactant or emulsifying agent can be added to enhance the stability of the emulsion. A variety of surfactants may be incorporated in this process, preferably to an amount between 0.1 and 5% by weight. Exemplary emulsifiers or surfactants which may be used include most physiologically acceptable emulsifiers, for instance egg lecithin or soya bean lecithin, or synthetic lecithins such as saturated synthetic lecithins, for example, dimyristoyl phosphatidyl choline, dipalmitoyl phosphatidyl choline or distearoyl phosphatidyl choline or unsaturated synthetic lecithins, such as dioleyl phosphatidyl choline or dilinoleyl phosphatidyl choline. Other hydrophobic or amphipathic compounds can be used in place of the phospholipid, for example, cholesterol. Emulsifiers also include surfactants such as free fatty acids, esters of fatty acids with polyoxyalkylene compounds like polyoxpropylene glycol and polyoxyethylene glycol; ethers of fatty alcohols with polyoxyalkylene glycols; esters of fatty acids with polyoxyalkylated sorbitan; soaps; glycerol-polyalkylene stearate; glycerol-polyoxyethylene ricinoleate; homo- and co-polymers of polyalkylene glycols; polyethoxylated soya-oil and castor oil as well as hydrogenated derivatives; ethers and esters of sucrose or other carbohydrates with fatty acids, fatty alcohols, these being optionally polyoxyalkylated; mono-, di- and tri-glycerides of saturated or unsaturated fatty acids, glycerides of soya-oil and sucrose. Other emulsifiers include natural and synthetic forms of bile salts or bile acids, both conjugated with amino acids and unconjugated such as taurodeoxycholate and cholic acid.

C. Effective Dosages

As used herein, the term “therapeutically effective amount” means the amount needed to achieve the desired therapeutic or diagnostic effect or efficacy when administered to the respiratory tract of a subject in need of treatment, prophylaxis or diagnosis. The actual effective amounts of drug can vary according to the biological activity of the particular compound employed; specific drug or combination thereof being utilized; the particular composition formulated; the mode of administration; the age, weight, and condition of the patient; the nature and severity of the symptoms or condition being treated; the frequency of treatment; the administration of other therapies; and the effect desired. Dosages for a particular patient can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to an individual is sufficient to effect a beneficial therapeutic response in the individual over time, or, e.g., to reduce symptoms, or other appropriate activity, depending on the application. The dose is determined by the efficacy of the particular formulation, and the activity and stability of the NE-targeting agent employed and the condition of the individual, as well as the body weight or surface area of the individual to be treated. The size of the dose is also determined by the existence, nature, and extent of any adverse side-effects that accompany the administration of a particular vector, formulation, or the like in a particular individual.

Formulations are administered at a rate determined by the median lethal dose (LD₅₀) of the relevant formulation, and/or observation of any side-effects of the compositions at various concentrations, e.g., as applied to the mass and overall health of the individual. Administration can be accomplished via single or divided doses. The necessary dose of the disclosed compositions will normally be determined by a physician, depending on the severity of the disease, medical history of the patient, and other complications that may be present. In one embodiment, the dose of heparin, heparin derivative, or fragment thereof prescribed may be from 0.01 mg to 5 g, more preferably from 0.05 mg to 1 g, still more preferably from 0.1 mg to 1 mg. These doses will typically be given once, twice or three times a day, and will preferably be given every 2 days, and more preferably every week. The length of treatment can range from two weeks, a month, six months, a year or longer. In some cases, the subject will remain on medication for an extended period of time. The regime of treatment may be adjusted according to the severity and progression of the condition.

D. Combination Therapy

The disclosed compositions can be administered alone, or in combination with one or more additional therapeutic, prophylactic or diagnostic agents. The one or more additional therapeutic agents may be used together or sequentially. In preferred embodiments, the compositions are supplemented with conventional treatments for chronic respiratory inflammation, including bronchiectasis, COPD, and bacterial and viral infections, or symptoms thereof. Conventional therapies for managing the symptoms of COPD, include, but are not limited to, protease inhibitors, anti-elastases, anti-inflammatories, mucolytics, antibiotics, antivirals, bronchodilators such as β2 agonists (e.g. salbutamol, albuterol, terbutaline), anticholinergics (e.g. ipratropium), and theophylline, and corticosteroids. In a preferred embodiment, the formulation is administered in combination with a protease inhibitor or anti-elastase, such as alpha-1-antitrypsin (α₁AT). This may be particularly desirable if the NE-targeting agent results in disruption or dissociation of NE binding to Syn-1. The addition of exogenous protease inhibitors or anti-elastases can speed the inhibition or degradation of neutrophil elastase. The disclosed composition may also be administered in combination with other therapeutic interventions such as supplemental oxygen, conventional pulmonary rehabilitation, nutritional modifications, or as an adjunct to surgery.

E. Storage and Kits

For storage and shipping, the disclosed formulations can be dissolved in a suitable solvent (e.g., an aqueous medium such as sterile water, and stored for long periods of time prior to use). Preferably, formulations including an NE-targeting agent are stored as a dry powder. For example, heparin or its fragments can also be dissolved in water, stored frozen and thawed for use, for instance in a nebulizer.

Kits containing formulations are disclosed. Kits contain one or more of the disclosed compositions and optionally include one or more of the following: bioactive agents, media, excipients, and a sterile vessel. The formulations can be in solution or dry (e.g., as a dry powder). Components of the kit may be packaged individually and are sterile. The compositions may be provided in dosage form, in containers including but not limited to, a graduated storage container for solutions, or blister pack for dry powder. Kits for intranasal and respiratory administration may optionally contain a delivery device for facilitating delivery, such as a nasal sprayer, metered dose inhalers (MDI), or nebulizer. The kits are generally provided in a container, e.g., a plastic, cardboard, or metal container suitable for commercial sale. Any of the kits can include instructions for use.

### III. Methods of Use

A. Methods of Administration

Delivery of NE-targeting agent formulations as described herein, have prophylactic and therapeutic application for a wide range of respiratory and lung diseases. Respiratory administration can typically be completed without the need for medical intervention (self-administration), the pain often associated with injection therapy is avoided, and the amount of enzymatic and pH mediated degradation of the bioactive agent, frequently encountered with oral therapies, is significantly reduced.

The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption into the circulating blood occurs. The alveoli are covered by a thin epithelium without cilia or a mucus blanket and secrete surfactant phospholipids (J. S. Patton & R. M. Platz. Adv. Drug Del. Rev. 8:179-196 (1992)). The drug is not intended for absorption into the circulating blood, but is targeted to inflamed sites of the airway epithelium

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung (Gonda, I. “Aerosols for delivery of therapeutic an diagnostic agents to the respiratory tract,” in Critical Reviews in Therapeutic Drug Carrier Systems, 6:273-313 (1990)).

Inhaled aerosols have been used for the treatment of local lung disorders including asthma and cystic fibrosis (Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)). Considerable attention has been devoted to the design of therapeutic aerosol inhalers to improve the efficiency of inhalation therapies (Timsina et. al., Int. J. Pharm., 101: 1-13 (1995); and Tansey, I. P., Spray Technol. Market, 4: 26-29 (1994)).

Aerosol dosage, formulations and delivery systems may be selected for a particular therapeutic application, as described, for example, in Gonda, I. “Aerosols for delivery of therapeutic and diagnostic agents to the respiratory tract,” in Critical Reviews in Therapeutic Drug Carrier Systems, 6:273-313, 1990; and in Moren, “Aerosol dosage forms and formulations,” in: Aerosols in Medicine, Principles, Diagnosis and Therapy, Moren, et al., Eds. Esevier, Amsterdam, 1985. The term aerosol as used herein refers to any preparation of a fine mist of particles, which can be in solution or a suspension, whether or not it is produced using a propellant. Aerosols can be produced using standard techniques, such as ultrasonication or high pressure treatment.

In preferred embodiments, the compound is administered through inhalation in a form such as liquid particles and/or solid particles. Suitable examples include, but are not limited to, a dry powder, an aerosol, a nebula, a mist, an atomized sample, and liquid drops. Typical apparatus which may be used for administration to humans include metered dose inhalers (MDI), nebulizers, and instillation techniques. The formulation is administered in an amount effective to treat or prevent, one or more symptoms or manifestations of an inflammatory disease, particularly a chronic respiratory inflammation. In the most preferred embodiment, NE-targeting agent optionally including a carrier is administered as dry powder using a dry powder inhaler, where the particles dissolve in the respiratory tract, for example within the lung secretions.

Various suitable devices and methods of inhalation which can be used to administer particles to a patient's respiratory tract are known in the art. Nebulizers create a fine mist from a solution or suspension, which is inhaled by the patient. The devices described in U.S. Pat. No. 5,709,202 to Lloyd, et al., can be used. An MDI typically includes a pressurized canister having a meter valve, wherein the canister is filled with the solution or suspension and a propellant. The solvent itself may function as the propellant, or the composition may be combined with a propellant, such as freon. The composition is a fine mist when released from the canister due to the release in pressure. The propellant and solvent may wholly or partially evaporate due to the decrease in pressure.

The compositions are preferably delivered into the respiratory tract with a pharmacokinetic profile that results in the delivery of an effective dose of the NE-targeting agent. As generally used herein, an “effective amount” of a NE-targeting agent is that amount which is able to, reduce or treat one or more symptoms of an inflammatory disease, reverse the progression of one or more symptoms of an inflammatory disease, halt the progression of one or more symptoms of an inflammatory disease, prevent the occurrence of one or more symptoms of an inflammatory disease, decrease a manifestation of the disease, as compared to a matched subject not receiving the composition. In a preferred embodiment, the inflammatory disease is chronic respiratory inflammation. Treatment of chronic respiratory inflammation with an effective amount of the disclosed compositions may improve symptoms including, by not limited to, inflammation, airflow restriction, chronic cough, sputum production, hemoptysis, dyspnea, air trapping, wheezing, chest pain and recurrent lung infection. In preferred embodiments, the composition also lowers NE activity and inflammatory cell count, as well as improves the lung tissue integrity.

As described above, the actual effective amounts of drug can vary according to the specific drug or combination thereof being utilized, the particular composition formulated, the mode of administration, and the age, weight, condition of the patient, and severity of the symptoms or condition being treated.

One or more of these compositions can be administered to an animal (e.g., a human) to modulate the activity of NE. A physician may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

B. Patients and Diseases to be Treated

The compositions are administered to a patient in need of prophylaxis or treatment of an inflammatory disease or disorder characterized by aberrant expression of NE. In a preferred embodiment, the disease or disorder is characterized by chronic respiratory inflammation, for example, bronchiectasis, emphysema, or chronic obstructive pulmonary disease. The compositions can be administered to animals or humans. The inflammatory diseases may be a respiratory tract or lung infection, a disease of the interstitium, or a disease of the airways. As used herein, a “lung cancer” refers to either a primary lung tumor (for example, bronchogenic carcinoma or bronchial carcinoid) or a metastasis from a primary tumor of another organ or tissue (for example, breast, colon, prostate, kidney, thyroid, stomach, cervix, rectum, testis, bone, or melanoma). As used herein, a “respiratory tract or lung infection” refers to any bacterial, viral, fungal, or parasite infection of any part of the respiratory system. As used herein, a “disease of the interstitium” includes any disorder of the interstitium including fibrosis (for example, interstitial pulmonary fibrosis, interstitial pneumonia, interstitial lung disease, Langerhans' cell granulomatosis, sarcoidosis, or idiopathic pulmonary hemosiderosis). As used herein, a “disorder of gas exchange or blood circulation”, refers to any abnormality affecting the distribution and/or exchange of gases to/from the blood and lungs (for example, pulmonary edema, pulmonary embolism, respiratory failure (e.g., due to weak muscles), acute respiratory distress syndrome, or pulmonary hypertension). As used herein, a “disease of the airway” includes any disorder of regular breathing patterns, including disorders of genetic and environmental etiologies (for example, asthma, chronic bronchitis, bronchiolitis, cystic fibrosis, bronchiectasis, emphysema, chronic obstructive pulmonary disease, diffuse panbronchiolitis, or lymphangiomyonatosis).

Representative respiratory diseases for treatment, or prevention of at least one symptom of the respiratory disease, include, but are not limited to, lung disease secondary to collagen vascular diseases such as systemic lupus erythematosis; rheumatoid arthritis; scleroderma; dermatomyositis; mixed connective tissue disorder; vasculitis associated lung disease such as Wegener granulomatosis and Good-pasture's Syndrome; sarcoid; and the syndrome of Acute Lung Injury/Acute Respiratory Distress Syndrome. The inflammatory component of these diseases involves some degree of autoimmunity, however the disclosed treatment does not address the autoimmune reaction.

In a preferred embodiment, compositions including one or more NE-targeting agents are administered to a subject suffering from, or at the risk of developing, chronic respiratory inflammation. A number of diseases including bronchiectasis and COPD are characterized by chronic respiratory inflammation. As chronic respiratory inflammation is a progressive disease, the subjects may be treated with the disclosed formulations at any stage of the disease. Subjects affected by chronic respiratory inflammation may display one or more of the following features or symptoms: prolonged and abnormal inflammation, permanently dilated bronchi, airflow limitation, chronic cough, sputum production, hemoptysis, dyspnea, air trapping, wheezing, chest pain and recurrent lung infection. In addition to clinical characteristics, subjects with chronic respiratory inflammation may be identified with the help of chest CT scan, chest x-ray, sputum culture, bronchoscopy and lung function tests.

The subject may be a tobacco smoker, either currently or formerly. In some cases, the subject may have been exposed to second-hand smoke. In addition, the subject may have been exposed to other pollutants, such as particulates emitted by industrial plants and factories, dust from mines and construction sites, and exhaust gas from vehicles. The subject may have a genetic disorder, e.g. alpha-1 antitrypsin deficiency, which predisposes to the development of chronic respiratory inflammation. The subject may also have a history of severe respiratory infection.

The lungs and airways of the subject in need of the disclosed compositions may have increased numbers of inflammatory cells, predominantly neutrophils. The subject may have an elevated level of NE, for example a molar excess of NE relative to alpha-1 antitrypsin. The subject may have elevated myeloperoxidase activity or exhibit airspace enlargement relative to a normal subject. Other inflammatory cells may also be present or elevated, including macrophages, basophils and eosinophils. The levels of mediators and chemoattractants such as tumor necrosis factor-α (TNF-α) and interleukin-8 (IL-8) may be present in elevated levels in subjects of chronic respiratory inflammation. Proteases, such as metalloproteases, elastases and cathepsins may also be found in increased levels. Therefore, treatment with the disclosed compositions will preferably reduce, inhibit, or decrease one or more of the following: the activity of NE, number of inflammatory cells and inflammation.

The present invention will be further understood by reference to the following non-limiting examples.

### EXAMPLES

### Example 1

### Generation of Non-Anticoagulant Heparin

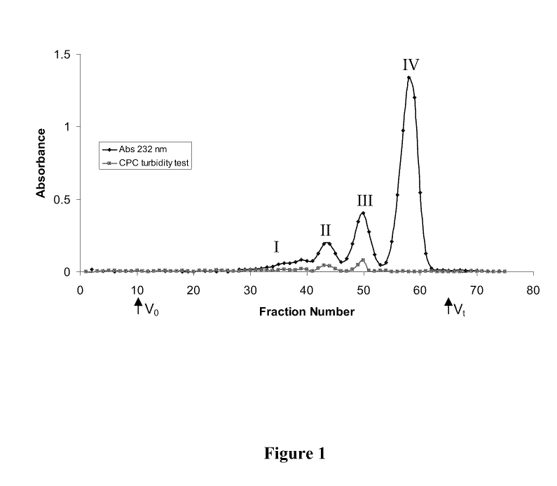

Commercially available porcine mucosal heparin was digested with heparitinase-III into smaller heparin fragments. The enzyme digestion product was then sorted according to their hydrodynamic size by gel filtration (FIG. 1). Four peaks were obtained and determined to contain di-, tetra-, hexa- and octasaccharides respectively as shown in Table 1, below. The length of heparin saccharide was determined by dividing the total heparin concentration (measured by 232 nm absorbance) by the concentration of glucuronic acid (GlcA) (measured by carbazole assay).

TABLE 1
Characterization of heparin oligosaccharides
following digestion with heparitinase-III

| | Conc. of heparin | | No. of GlcA per chain | |
|---|---|---|---|---|
| | oligosaccharides | Conc. of | (GlcA conc./heparin | Length of heparin |
| | (mM) | GlcA (mM) | oligosaccharide conc.) | oligosaccharide |
| Peak I | 13.2 | 65.3 | 4.9 | Octa & higher |
| Peak II | 34.1 | 106.2 | 3.1 | Hexa |
| Peak III | 36.0 | 94.3 | 2.6 | Tetra |
| Peak IV | 97.8 | 194.6 | 0.9 | Di |

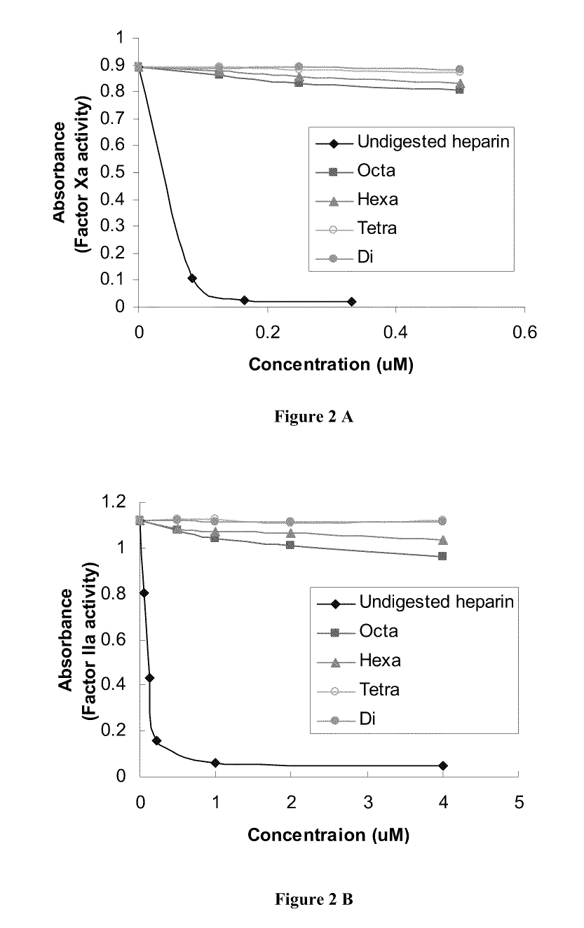

All heparin fragments exhibited negligible anticoagulant activity (FIG. 2) as they induced little inhibition of coagulation factors Xa and IIa, when compared with full-length heparin in factor Xa (FIG. 2 A) and factor IIa (FIG. 2 B) assays.

### Example 2

### Preparation of Chitosan Beads Loaded with Heparin Fragments

Five-micron (5 μm) chitosan beads were prepared by SPG membrane emulsification technique as described in Wang, et al., J Control. Release 106:62-75 (2005). Briefly, chitosan solution was prepared by dissolving 1.6% of chitosan into a 1% (w/v) acetic acid and 5% (w/v) sodium chloride. 2 ml of chitosan solution was then poured into the Teflon tank as the dispersed phase (aqueous phase) and allowed to pass through the SPG membrane (pore size=5 μm) into continuous oil phase with 4% (w/v) Tween® 80 as emulsifiers, under pressure (16 Kpa) and at 600 rpm to form W/O emulsion for 2 hrs. Chitosan beads were solidified by addition of sodium hydroxide mixture containing 100 μl of 10M NaOH, 100 μl of Tween® 80 and 4 ml of oil. Chitosan beads were then collected and washed 2 times with 1% Tween 80 followed by 3 times with distilled water. The chitosan beads were then air dried and stored at room temperature. 9.8 ng of heparin fragment was loaded onto 1 mg chitosan bead by incubating the heparin fragments (10 μg/ml, product in peak I which contains octa- and higher saccharides) with chitosan beads (50 mg) at pH 4.8 at 37° C. for 18 hours.

### Example 3

### Heparin Derivative-Loaded Chitosan Treatment Reduces the Immune Response in Rats Exposed to Cigarette Smoke

Materials and Methods

### Smoking Rat Model

Sprague-Dawley rats were divided into two groups, the cigarette smoke group and the sham air group. The cigarette smoke group (8 rats) was exposed to smoke from 11 cigarettes in a ventilated smoking chamber (1 hr per day for 4 weeks). The sham air group (8 rats) was placed in the apparatus for the same period of time and exposed to room air instead. After 4 weeks of cigarette smoke or sham air exposure, four rats from each group were treated with 2 mg of heparin derivative-loaded chitosan (Hp-chitosan). Each rat was approximately 400 g. The rats were anaesthetized with pentobarbitone, and 2 mg Hp-chitosan/rat was administered intratracheally with DP-4 dry powder insufflator™ device (PennCentury Inc., Philadelphia, Pa., USA). The remaining four rats in each group were given a similar dose of neat chitosan beads as vehicle control. The rats were allowed to recover from the anesthetics. After 72 hours, the rats were sacrificed by an overdose of pentobarbitone. The bronchoalveolar lavage fluid (BALF) and the lung tissue were collected and analyzed.

### Analysis of Cells in BALF

The cells in BALF were diluted to a concentration of 2×10⁵ cells/ml. 0.2 ml of cell suspension was then spun down onto a poly-L-lysine coated glass slide at 1000 rpm for 5 min using a Cytospin 2 cytocentrifuge (Shandon Instruments, Sewickley, Pa.). The slides were fixed and stained using May-Giemsa method. The cells on the slides were then counted under the microscope.

### Determination of Tissue Integrity

Tissue integrity was determined in terms of airspace enlargement, which was measured using a modified method as described in Chan, et al. Respir Med. 103: 1746-1754 (2009). Images of 10 fields for each lung section were captured randomly at ×40 magnification. Quantification of airspace enlargement in each tissue section was determined by mean linear intercept (Lm). The measurement of Lm was performed by means of a 100×100 μm grid passing randomly through the lung section. The total length of each line of the grid divided by the number of alveolar intercepts gave the Lm.

Results

The total cell count increased significantly in cigarette smoke-exposed rats when compared with rats from the sham air group. It is believed this is due to the large-scale migration of inflammatory cells in response to cigarette smoke. Among the inflammatory cells, the percentage of macrophages and neutrophils increased in particular. These cells release potent enzymes upon stimulation, which may cause tissue damage in the local environment especially when inadequately inhibited by their inhibitors. Hp-chitosan treatment effectively lowered total cell count.

TABLE 2
Analysis of cells collected from bronchoalveolar lavage fluid (BALF) in treatment
and control groups for cigarette smoke-exposed, and sham air rats

| | sham air + | sham air + | smoking + | smoking + |
|---|---|---|---|---|
| | Chitosan | Hp-chitosan | Chitosan | Hp-chitosan |
| Total cells (10⁵/ml) | 2.55 ± 0.13 | 2.73 ± 0.33 | 9.13 ± 0.43 | 5.35 ± 0.24 |
| Neutrophils (%) | 7.73 ± 0.22 | 7.38 ± 0.17 | 19.58 ± 0.75 | 11.85 ± 0.52 |
| Eosinophils (%) | 2.55 ± 0.13 | 2.95 ± 0.57 | 0.75 ± 0.13 | 1.00 ± 0.18 |
| Lymphocytes (%) | 24.53 ± 2.86 | 23.70 ± 3.90 | 8.73 ± 1.44 | 18.98 ± 1.96 |
| Macrophages (%) | 65.18 ± 2.73 | 65.25 ± 2.02 | 70.95 ± 0.70 | 68.18 ± 1.95 |
| Protein (mg/ml) | 0.14 ± 0.04 | 0.14 ± 0.07 | 1.08 ± 0.61 | 0.71 ± 0.12 |

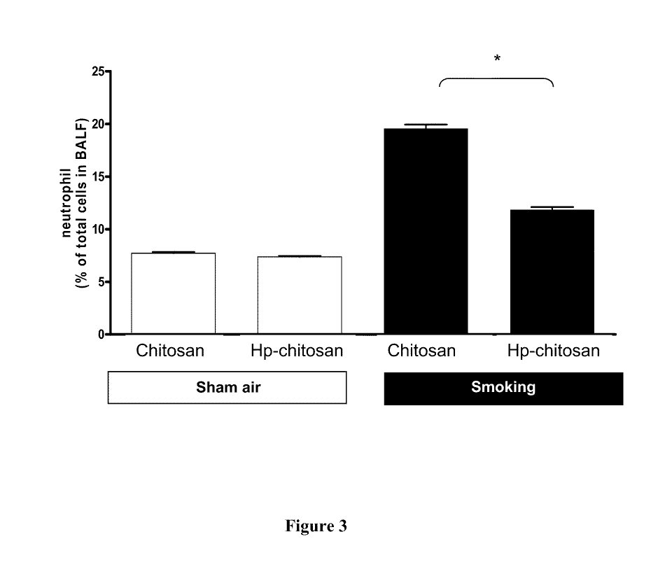

As shown in Table 2 (above) and FIG. 3, Hp-chitosan effectively lowered the mean percentage of neutrophils. Neutrophils are a crucial participant of the secondary host defense system. They engulf and degrade microorganisms by releasing reactive oxygen species, antimicrobial peptides and proteases. The neutrophils were identified morphologically by their multi-lobed nuclei as revealed by the May-Giemsa stain. The mean percentage of neutrophils among other cell types in BALF in rats from the cigarette smoke group markedly increased when compared with the sham air group. With Hp-chitosan treatment, cigarette smoke-exposed rats exhibited a much lower percentage of neutrophils than those receiving the empty vehicles (P less than 0.001).

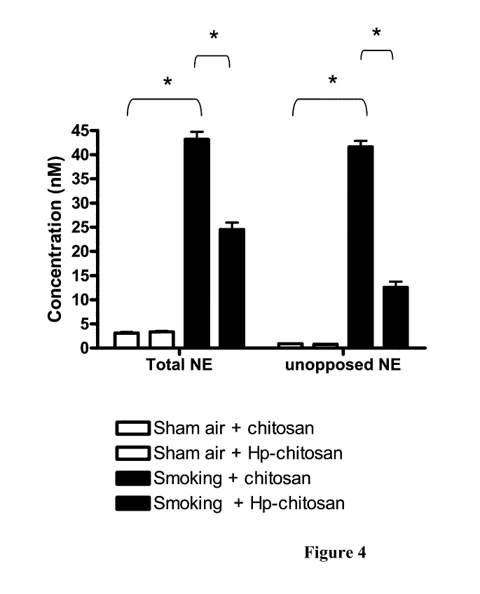

As shown in FIG. 4, reduction in unopposed neutrophil elastase (NE) concentration was observed in cigarette smoke-exposed rats treated with Hp-chitosan. NE is a potent protease released by neutrophils. It digests a wide range of substrates, including many structural proteins found in the airways and lungs. Therefore, NE can cause extensive damage if its activity is not properly controlled by its inhibitors. NE concentration was determined from the bronchoalveolar lavage fluid using 2 mM MeO-Suc-Ala-Ala-Pro-Val-p-nitroanilide, an NE-specific substrate, in 0.2 M Tris-HCl, 0.5 M NaCl, pH 8.0. NE catalyzes the hydrolytic release of p-nitroaniline, which was then measured by absorbance at 410 nm, 37° C. (E₄₁₀=8800) using a spectrophotometer. The NE activity of the BALF was then determined with reference to the activity of purified, commercially available NE, of which the activity was confirmed by active site titration. A dramatic increase in total NE concentration was observed in rats from the cigarette smoke group compared to those from the sham air group. The majority of NE present was unopposed, meaning that the action of NE inhibitors were inadequate. However, with the treatment of Hp-chitosan, both total and unopposed NE concentrations were effectively lowered (P less than 0.001).

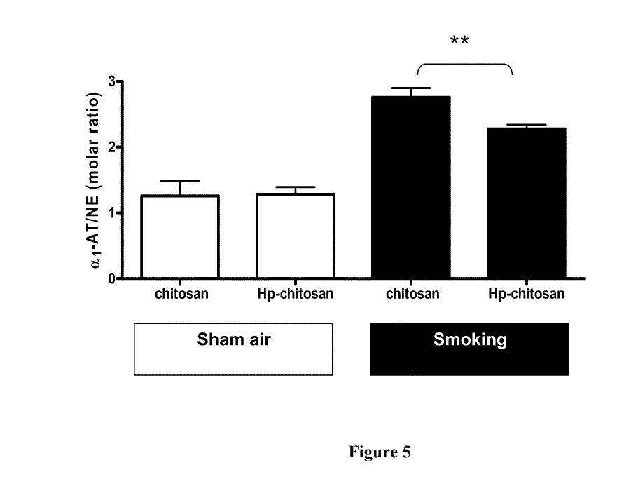

Hp-chitosan treatment partly relieved the molar excess of alpha-1-antitrypsin (α₁-AT) in relation to NE (FIG. 5). In the healthy airways and lungs, the molar ratio between α₁AT and NE is about 1:1, such that NE activity is effectively controlled without excessive production of α₁AT. However, in patients with chronic respiratory inflammation, as well as rats of the cigarette smoke group, α₁AT /NE ratio is very elevated. This is due to the inaccessibility of NE as a result of partnering with a glycoprotein called syndecan-1, as described in Chan, et al., Am J Respir Cell Mol Biol. 41: 620-628 (2009). The body responded to the persistent NE activity by producing more α₁AT. However, despite the overwhelming excess of α₁AT, NE remained largely active. NE thus continually acts on proteins in the airway and lung environment, leading to injury. As shown in FIG. 5, Hp-chitosan treatment resulted in a lowered α₁AT /NE ratio (P less than 0.01). It is therefore believed that it can, at least partly, return NE to a state accessible to α₁AT.

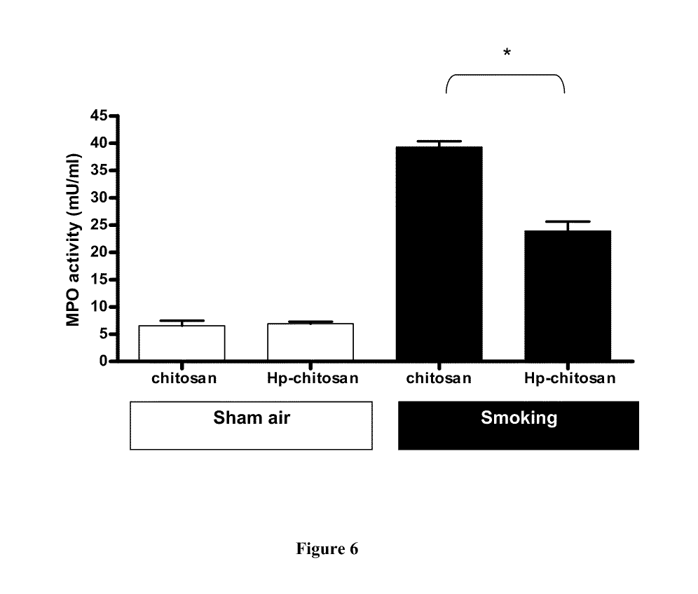

Hp-chitosan also lowered myeloperoxidase (MPO) activity in BALF of rats from the cigarette group (FIG. 6). MPO is a peroxidase found in neutrophils. It produces radicals for the elimination of micro-organisms. MPO activity of BALF was assayed spectrophotometrically using 0.0005% hydrogen peroxide as substrate in 50 mmol/L phosphate buffer containing 0.167mg/ml o-dianisidine dihydrochloride at pH 7.0, 37° C. The change in absorbance at 460 nm was measured using a spectrophotometer. The MPO activity was then determined with reference to purified, commercially available MPO, of which the activity was verified by active site titration. MPO activity was significantly higher in BALF of cigarette smoke-exposed rats when compared with those of the sham air group. As shown in FIG. 6, a decrease in MPO activity was observed when cigarette smoke-exposed rats were treated with Hp chitosan (P less than 0.001).

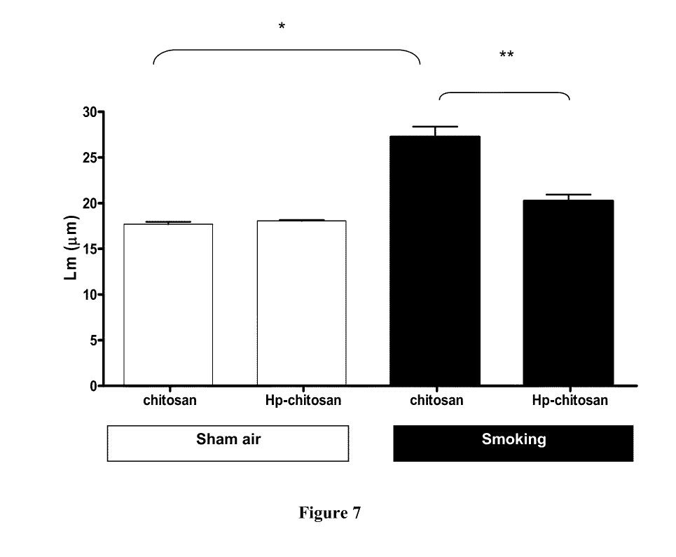

Airspace enlargement, a measure of tissue integrity, was reduced in cigarette smoke-exposed rats with Hp chitosan treatment (FIG. 7). More prominent airspace enlargement, measured as described above, was observed in cigarette smoke-exposed rats when compared with the sham air control rats. The mean linear intercept (Lm) of the smoke exposed rats were significantly higher than that of the sham air rats (27.25±2.3 μm vs 17.71±0.5 μm) (P less than 0.001). As shown in FIG. 7, a reduction in Lm was observed when cigarette smoke exposed rats were treated with Hp chitosan (27.25±2.3 μm vs 22.98±1.6 μm) (P less than 0.01).

Hp-chitosan treatment lowered NE staining in lung specimen from rats of the cigarette smoke group. Paraffin embedded lung sections from rats of the cigarette smoke group and sham air controls were analyzed by immunohistochemistry. Sections from lungs of rats exposed to cigarette smoke displayed more intense NE-staining and poorer tissue integrity when compared with sections from the sham air group. With Hp-chitosan treatment, the number of NE-positive cells and the size of the airspace decreased. Among the sham air group, those receiving chitosan and Hp-chitosan exhibited some staining for NE when compared with specimen from animals receiving neither the loaded nor the empty carrier (E). It is believed this is due to an inflammatory response elicited by the intratracheal administration procedure.

### Example 4

### Affinity of α₁-AT for the Syn-1 Core Protein in Bronchiectasis Patients

Bronchiectasis is a chronic pulmonary disease characterized by irreversibly dilated bronchi that are inflamed and colonized by bacteria. Affected patients suffer from chronic sputum production and recurrent exacerbation. Progressive bronchial injury and deterioration eventually culminate in respiratory failure. The intense but localized inflammation is dominated by neutrophils, which undergo degranulation upon cell activation, phagocytosis, or cell death in the airways. Neutrophil elastase (NE) is released as a 29-kD active enzyme in the degranulation product. In the airways, NE activity is countered by secretory leukoprotease inhibitor, locally produced by submucosal glands of the bronchial epithelium. If local production of secretory leukoprotease inhibitor is overwhelmed, plasma-derived α₁-AT becomes the major antiprotease defense. Deficiency in α₁-AT has been held the cause of protease/antiprotease imbalance in the airways of patients with chronic obstructive pulmonary disease, emphysema, and cystic fibrosis. Less is known about inflammatory environments where NE activity remains unopposed, as in bronchiectasis. In the bronchial secretions, NE that is complexed with syndecan (Syn)-1 becomes inaccessible to serpins. Similarly, in vitro studies show that α₁-AT inactivation of NE is limited when NE is bound to polyanions, such as DNA and glycosaminoglycans.

Syndecans are integral membrane proteoglycans, which bear heparan sulfate (HS) and chondroitin sulfate (CS) moieties in ectodomains of the core proteins. The HS moiety consists primarily of linear disaccharide repeats of GlcA and GlcNAc, modified to different degrees by N-sulfation and O-sulfation. It is unclear how HS biosynthesis is regulated to result in highly sulfated domains interspersed with less sulfated ones along the sequence of disaccharide repeats. The structural diversity, however, provides the HS moiety with wide-ranging affinities for binding partners. As a result, the HS moiety confers on the ectodomain of membrane-bound Syn important roles in the regulation of pericellular activities.

Syndecan ectodomain shedding can be mediated by tissue inhibitor of metalloproteinase (TIMP)-3-sensitive metalloprotease and bacterial proteases, as shown in cell culture and mouse models. Secretions of inflamed airways and skin wounds indicate NE and anti-elastases in supramolecular complexes with shed Syn-1. Given that the HS moiety remaining on the shed ectodomain shares polyanionic characteristics of heparin and DNA, it was hypothesized that the HS moiety of Syn-1 binds NE and modulates effects of anti-elastases in the inflammatory environment. To demonstrate that the HS moiety was responsible for NE binding, release of NE from the sputum complex after HS cleavage at heparitinase (HS^{ase}) accessible sites in the complex was tested. Recombinant human Syn (rhSyn)1 was exploited to demonstrate that Syn binding to NE was sufficient to interfere with α₁-AT inhibition of NE activity, mimicking observations made on the sputum complex. Together with assays of NE-HS affinity, the data confirm that the HS moiety of Syn-1 binds NE, and that the binding interferes with the anti-elastase function of serpin in chronic bronchial inflammation.

Materials and Methods

Subjects and Sputum Samples

Patients with bronchiectasis were recruited from the Bronchiectasis Clinic, The University of Hong Kong, Queen Mary Hospital. Inclusion criteria were: bronchiectasis documented on high-resolution computed tomography of chest; idiopathic etiology of bronchiectasis; chronic sputum production, with daily sputum greater than 10 ml; absence of asthma (according to American Thoracic Society guidelines) and other major pulmonary diagnoses; steady state as defined by absence of change of symptoms noted by the patient over the past 3 weeks. Exclusion criteria were: bronchiectasis with defined etiology (e.g., post-tuberculous, primary ciliary dyskinesia; common variable immunodeficiency); maintenance use of oral or nebulized antibiotics; use of antibiotics within the previous 3 weeks. A sweat test was not performed to exclude cystic fibrosis, because of the known rarity of cystic fibrosis among Chinese individuals and the lack of suggestion of multisystem disease in any of the patients. The study was approved by the University of Hong Kong Ethics Committee; patients gave informed consent before sputum collection.

Assay for NE Activity/Concentration

Unopposed NE activity in sputum sol was assessed for equivalent molar concentration by active-site titration. The NE activity of sputum sol was estimated using MeO-Suc-Ala-Ala-Pro-Val-p-nitroanilide as substrate at 2 mM in 0.2 M Tris-HCl, 0.5 M NaCl (pH 8.0); the hydrolytic release of p-nitroaniline was followed at 410 nm, 37° C. (molar extinction coefficient at 410 nm=8,800). An equivalent activity of purified human NE (Sigma, St. Louis, Mo.) was titrated with increasing concentrations of the irreversible inhibitor, MeO-Suc-Ala-Ala-Pro-Val-CMK (Calbiochem, La Jolla, Calif.), and the residual activities were measured. Extrapolation of the plot of residual activity against molar concentration of inhibitor to zero activity yields the concentration of active-site—titrated NE. The concentration of α₁-AT (human placenta; Sigma) was standardized against the active-site-titrated human NE. This formed the basis for standard additions of anti-elastases to incubations of active-site—titrated NE in the presence of sputum Syn-1 or rhSyn-1 for inhibition of NE activity.

Assay for Total NE Concentration in Sputum Sol

The total protein level of NE in the sputum sol phase was measured with a quantitative sandwich enzyme-linked immunosorbent assay (ELISA) kit (Bender MedSystems, Austria) according to the manufacturer's instructions.

Assay for α₁-AT Concentration

Sandwich enzyme-linked immunosorbent assay (ELISA) for α₁-AT level in sputum sol was performed with goat anti-human α₁-AT (Sigma) immobilized in the wells of microtitre plates. Non-specific binding was blocked with 3% bovine serum albumin. Known dilutions of sputum sol were applied at 100 μl/well and incubated (1 h, 37° C.) to allow α₁-AT The captured antigen was incubated (1 h, 37° C.) in turn with the primary antibody, rabbit anti-human α₁-AT (diluted 1:400, Roche Molecular Biochemicals, Indianapolis, Ind.) (50 μl), secondary antibody, goat anti-rabbit IgG conjugated with peroxidase (diluted 1:1000; Roche) (50 μl) and the substrate (o-phenylenediamine, Sigma) for color development. Wells were washed with PBS-Tween® 20 between incubations. Absorbance at 490 nm was read with a microplate reader (Molecular Devices Corporation, Sunnyvale, Calif.). Assays were performed in duplicate. The standard curve was prepared with serial dilutions of reference α₁-AT (human placenta; Sigma). Standard additions of reference α₁-AT to defined dilutions of a test sputum sol results in parallel upward shift in the standard curve, indicating little loss of α₁-AT during incubations with sputum samples.

Deglycanation of Syns

Sputum sol was treated (30 min, 24° C.) with the mucolytic reducing agent, DTT (5 mM; Sigma) to deactivate NE, dialyzed against PBS (16 h, 4° C.) to remove DTT, and then incubated (16 h, 37° C.) with HS^{ase} I and II (0.01 unit each; Seikagaku, Tokyo, Japan) or chondroitinase ABC (0.01 unit; Seikagaku). Control treatments were performed in the relevant incubation buffer (product sheet; Seikagaku), but without the indicated enzymes. The digestion products were subjected to Western blot and Western ligand blot analyses. Recombinant Syn-1 was similarly treated, deglycanated, and analyzed, but without prior treatment with DTT.

Western Blot and Western Ligand Blot Analyses

Sputum sol samples were treated with DTT, as described above, and then subjected to SDS-PAGE in a 10% gel under nonreducing conditions. The gel electrophoretogram was electroblotted onto polyvinylidene fluoride membrane and nonspecific binding was blocked with 5% nonfat dry milk (1 h, 24° C.) before the blot was probed for the epitope of interest. Primary antibodies used were those against: α₁-AT (diluted 1:200, rabbit anti-human α₁-AT; Sigma); Syn-1 (diluted 1:200, mouse anti-human CD138; Serotec, Raleigh, N.C.); Syn-4 (diluted 1:200, mouse anti-human Syn-4; Santa Cruz Biotechnology, Santa Cruz, Calif.); NE (diluted 1:200, mouse anti-human NE; Dako, Glostrup, Denmark); 10e4 (diluted 1:400, mouse anti-HS; Seikagaku); and CS-56 (diluted 1:300, mouse anti-CS; Sigma). Secondary antibodies were conjugated with horseradish peroxidase. The membranes were washed with PBS-Tween® 20 between incubations. Visualization was enhanced with an enhanced chemiluminescence kit (Amersham Biosciences, Piscataway, N.J.). Bound antibodies were stripped from the membrane by incubation (30 min, 50° C.) with 100 mM 2-mercaptoethanol, 2% SDS, and 62.5 mM Tris-HCl (pH 6.7), before reprobing with another primary antibody. Alternatively, the Western blot was blocked and then incubated (1 h, 4° C.) with human NE or human α₁-AT (Sigma) as test ligand. The Western ligand blot was probed for the test ligand with the relevant primary and secondary antibodies, and visualized as described above.

Molecular Cloning of Human Syn-1

Total RNA was extracted from human bronchial epithelial cells (Clonetics, San Diego, Calif.) using the RNeasy Mini kit (Qiagen, Valencia, Calif.). Oligo-dT-primed first-strand cDNA was generated from the total RNA using SuperScript II reverse transcriptase (Invitrogen, San Diego, Calif.). The cDNA was used as template in a PCR to amplify the full-length coding sequence of Homo sapiens Syn-1 (NM_{—}002997; GenBank, National Center for Biotechnology Information, Bethesda, Md.) and to introduce Hind III and Xba I sites in the PCR product. Forward and reverse primers were, respectively, 5′-GCAAGCTTGAGAGCATCGAGC-3′(Hind III site in bold) and 5′-GCTCTA GACTCCCGCGTCAGG-3′ (Xba I site in bold). PCR was performed at 95° C. (12 min), then 30 cycles of 94° C. (1 min), 55° C. (1 min), and 72° C. (2 min), and finally 72° C. (10 min). The gel-purified PCR product was cloned into pGEM-T-Easy vector (Promega, Madison, Wis.), and then checked for the correct sequence with a Model 373A DNA sequencer (Applied Biosystems Inc., Foster City, Calif.). The human syndecan (hSyn)-1 sequence was excised from pGEM-T-Easy vector and then subcloned into expression vector, pcDNA3.1 (Invitrogen) using Hind III and Xba I. Recombinant plasmids were analyzed by restriction mapping to confirm the sense orientation of pcDNA3.1-hSyn-1.

Expression of Recombinant Human Syn-1 in Stable ARH-77 Transfectants

ARH-77 cells (human B-lymphoid cell line, CRL-1621; American Type Culture Collection, Rockville, Md.) were transfected with pcDNA3.1-hSyn-1 by FuGENE 6 Transfection Reagent (Roche Molecular Biochemicals, Indianapolis, Ind.) according to the manufacturer's instructions. Stable clones were selected at high concentration of G418 (500 mg/ml; Sigma) and maintained in RPMI 1,640 supplemented with 5% FBS, penicillin (100 U/ml), streptomycin (100 mg/ml), and G418 (400 mg/ml). Expression of Syn-1 was analyzed by dot blotting and immunocytochemistry using monoclonal antibody CD138 against human Syn-1 as described in the online supplement. The recombinant protein product, rhSyn-1, was then purified.

Cytocentrifugation and Immunocytochemistry

The transfected ARH-77 cells (2×10⁴ cells) were first washed twice in PBS and then cytocentrifugated (400 rpm, 5 min) onto poly-L-lysine coated slides by Cytospin® 3 cytocentrifuge (Shandon, Pittsburgh, Pa., USA). The cells were fixed in 4% (w/v) paraformaldehyde and permeabilised with 0.01% (v/v) Triton X-100. The slides were first incubated with primary antibody against syndecan-1 (diluted 1:100, mouse anti-human CD138; Serotec, Raleigh, N.C.), followed by biotinylated secondary antibody (diluted 1:200; Dako, CA) and streptavidin conjugated alkaline phosphatase (diluted 1:50; Dako). Visualization was performed by incubation of the slides with nitro-blue tetrazolium (NBT) and 5-bromo-4-choro-3-indolyl-phosphate (BCIP). The slides were mounted with Gel/mount™ (Biomeda, CA) and then observed under Olympus IX71 microscope system (Olympus, CA). Immunocytochemistry performed on cytospin preparations indicated that the syndecan-1 transfectants were CD138-positive but not the non-tranfected host cells.

Surface Plasmon Resonance Analysis of NE-HS Binding

Interactions of HS and NE were analyzed with a BIAcore 2,000 optical biosensor (BIAcore, Uppsala, Sweden). HS chains recovered from rhSyn-1 after LiOH treatment were biotinylated at the reducing ends (68 nmol biotin/mg HS, as determined by Quant*Tag Biotin kit; Vector Laboratories, Peterborough, UK). By a 2-minute injection of biotinylated HS, 50 mg/ml in 10 mM Hepes (pH 7.4), 0.15 M NaCl, and 0.005% P20 surfactant (HBS-P; BIAcore), at a flow rate of 10 ml/min onto the streptavidin (SA) sensor chip (BIAcore), increase in 250 resonance units indicated successful immobilization of HS onto the sensor chip. Increasing concentrations of NE in HBS-P buffer were perfused (at 10 ml/min for 2 min) in turn over the HS-SA sensor chip, each followed by perfusion of buffer to facilitate dissociation. Responses were monitored at 25° C. The sensor chip was regenerated with perfusion of 2 M NaCl/HBS-P. Resonance data obtained from sensorgrams were evaluated with BIAevaluation 3.0 software (BIAcore) to yield rate constants for association (kₐ), dissociation (k_{d}) and the equilibrium dissociation constant (K_{d}). For competition assays, NE (2,000 nM) premixed at 1:1 (vol/vol) with defined concentrations (250-2,000 ng/ml) of the competing glycosaminglycan-chondroitin 4-sulfate (C4S), heparin, and chemically modified heparins (N-desulfated re-N-acetylated, completely desulfated re-N-acetylated, or 2-O and 6-O desulfated and N-sulfated; Seikagaku) in HBS-P buffer for 30 minutes at 4° C. was injected onto the HS-SA sensor chip at 10 ml/min. Dissociation and regeneration procedures were as described above. Control runs with NE only (without competing glycosaminoglycan) were performed to ensure comparable results between test runs. All experiments were preformed in duplicate. Resonance data obtained from sensorgrams were plotted as percent inhibition relative to that obtained without competing glycosaminoglycan. The concentration of competing glycosaminoglycan that interfered with NE binding to the HS-SA sensor by 50% was determined using the statistical program, PRISM (GraphPad, Inc., San Diego, Calif.).

Spirometry Tests

The lung function tests included spirometric tests of forced vital capacity (FVC) and forced expiratory volume in 1 second (FEV1). Spirometry measurements were done using a Sensor-Medics 2200 Pulmonary Function Lab System (SensorMedics, Yorba Linda, Calif.) (E1). Sputum samples from patients with bronchiectasis were collected over a maximum of 4 h in sterile pots. Patients taking inhaled bronchodilators or inhaled corticosteroids had been advised to omit these drugs for at least 12 h before sputa were collected. Expectorated sputum samples were examined under light microscopy; presence of ≧10 buccal squamous epithelial cells per field (×100 magnification), as average of 10 fields was indicative of salivary origin. Such samples were excluded from further processing. Sputum samples were also sent for microbiological examination using standard techniques. The bulk of the collected sputum samples were immediately centrifuged at 50,000 g for 1.5 h (4° C.); the supernatant sol phase of each sample was collected, aliquoted and stored at −80° C. until use.

Statistical Analysis

Data are expressed as mean values (mean±SD). The INSTAT (GraphPad, Inc., San Diego, Calif.) and PRISM statistical software packages were used. Data were analyzed by one-way ANOVA with a Bonferroni post hoc test or Mann-Whitney U test. Differences are considered statistically significant with a P value of less than 0.05.

Results

Unopposed NE Despite Excesses of a1-AT in Sputum Sol

For this study, 12 subjects were recruited with non-cystic fibrosis bronchiectasis, proven by high-resolution computed tomography scan of the thorax. Unopposed NE concentrations in the sputum sol samples were determined by active-site titration, and these did not differ significantly from total NE concentrations, as determined by ELISA. This suggests that, in the majority of cases, NE exists in forms that are inaccessible to physiological anti-elastases. In this regard, concentrations of α₁-AT in the sputum sol samples were found to be in excess of unopposed NE concentrations at molar ratios that averaged 16:1. The results, therefore, reveal unopposed NE activity despite excesses of α₁-AT in the bronchial secretions of patients with bronchiectasis.

Unopposed NE Activity in the Sputum Complex Involves Syn-1, Not Syn-4

Unopposed NE in a supramolecular complex and binding of NE to Syn-1 in the complex interfered with anti-elastase action of a1-AT that was also associated with the complex. Syn-4 immunoreactivity was found in the molecular mass range of 31-50 kD, not in colocalization with endogenous NE. This contrasted with Syn-1 immunoreactivity in the high-molecular mass range of 100-250 kD, where colocalization with endogenous NE was observed. Western ligand blot with purified NE indicated capacity of the Syn-4-positive zone to bind NE. Thus, soluble forms of Syn-1 and -4 were both present in the sputum sol samples, and both forms showed capacities to bind NE. The observation of selective binding of endogenous NE to Syn-1 in the high-molecular mass sputum complex, however, suggests that NE encountered shed Syn-1 when neutrophils were activated as they coursed through the inflamed bronchial epithelium.

Release of NE from Sputum Syn-NE Complex

To demonstrate the importance of the HS moieties for binding, sputum sol samples were first treated with the reducing mucolytic agent, DTT, to inhibit NE activity, and then with HS^{ase} I and II to cleave HS. Western blot analysis of untreated control samples indicated the complexed form as a smear immunopositive for Syn-1, HS, CS, and NE at the nominal molecular size range of 250-100 kD. After treatment with DTT, small changes in mobility were observed, suggesting little effect on NE binding to the complex. Treatment with HS^{ase} I and II resulted in shift of the Syn-positive smear to a lower size range of 150-80 kD, complete loss of the HS epitope, and shift of the NE-positive smear to a lower size range (30-20 kD. Control treatment of the samples with chondroitinase ABC resulted in no mobility shift, as shown in blots probed for the Syn, HS, and NE epitopes, but loss of the CS epitope. The results, therefore, suggest that the HS moiety is responsible for binding of NE to Syn-1 in the sputum complex.

rhSyn-1 from ARH-77 Transfectants Bears HS and CS Moieties

To obtain adequate amounts of purified Syn-1 for study of NE binding and anti-elastase effects, stable transfection of ARH-77 cells with a pcDNA3.1-hSyn-1 construct was established. Dot blots probed with the monoclonal antibody CD138 against human Syn-1 indicated immunopositivity in lines derived from stable transfectants, but not in mock transfectants or non-transfected cells. Immunocytochemistry performed on cytospin preparations indicated that the Syn-1 transfectants were CD138-positive, but that the nontransfected host cells were not. A cellular source of rhSyn-1 was thus derived.

The purified rhSyn-1 was then analyzed for CS and HS moieties. Western blot analysis indicated a smear immunopositive for Syn-1, CS, and HS at the nominal molecular size range of 200-97 kD. Treatment with HS^{ase} I and II resulted in shifts of the Syn-1-positive smear to 97-70 kD, but decrease of the HS-positive smear to a band of approximately 70 kD. Treatment with chondroitinase ABC resulted in shift of Syn-1-positive smear to 180-90 kD, and loss of the CS epitope. The HS^{ase}-resistant HS epitope remained with the Syn-1 core protein after consecutive treatments with the enzymes. Results, therefore, indicate the presence of CS and HS moieties in the rhSyn-1 preparation.

The HS Moiety Binds NE and the Syn Core Protein Binds α1-AT

To test if binding of NE to Syn is indeed mediated by the HS moiety, rhSyn-1 was analyzed by Western blot, probed first for Syn-1, reprobed for HS, and then further probed for NE binding in Western ligand blot. The Western blots indicated mobility shift of the Syn-1/HS-positive smear, and significant loss of the HS epitope after HS^{ase} I and II treatment. The Western ligand blot demonstrated NE binding to rhSyn-1 that bore intact HS, but not after the HS^{ase}-susceptible HS moiety of rhSyn-1 had been cleaved. The results, therefore, indicate that NE binding to Syn-1 was mediated by the HS^{ase} susceptibleHS moiety.

To test if α₁-AT binds Syn, the protocol of Western blots was repeated for α₁-AT binding in Western ligand blot. The Western blots indicated the expected mobility shifts with heparitinase and chondroitinase treatments. The Western ligand blot demonstrated a ₁-AT binding to the deglycanated rhSyn-1 core protein, but not to the glycanated rhSyn-1. Taken together, the results suggest that, in the inflammatory environment where native Syn-1 can be variously deglycanated, the HS moiety of native Syn-1 varies in affinity for NE, whereas the core protein varies in affinity for α₁-AT.

To characterize NE-HS interactions, the HS moiety recovered from purified rhSyn-1 was immobilized on a sensor chip for surface plasmon resonance analysis. Sensorgrams of NE (50-1,500 nM) perfusion over the HS sensor chip yielded an equilibrium dissociation constant (K_{d}) of 14.17 nM, indication high affinity of NE for the immobilized HS. The kinetic data fitted to a 1:1 Langmuir binding model and the binding data are presented in Table 3. The values are in the same range as those reported for NE interaction with the ectodomain of Syn-1. To assess the affinity of NE for sulfation variants of heparin, the variants were premixed with NE before use in solution-surface competition experiments. With NE at 1,000 nM and competing glycosaminoglycans at 1,000 ng/ml, competitive effectiveness decreased in order of heparin (by 95%), N-desulfated and re-N-acetylated heparin (by approximately 75%), 2-0 and 6-0 desulfated and N-sulfated heparin (by approximately 60%). Completely desulfated and re-N-acetylated heparin, as well as C4S failed to outcompete NE binding to immobilized HS on the sensor chip. The corresponding concentration values of competing glycosaminoglycan that interfered with NE binding to the HS-SA sensor by 50% for heparin, its sulfation variants, and CS reflect the same gradation in binding affinity. The results indicate that the binding to NE is HS specific, and affinity is dependent on O-and N-sulfated domains along the HS chains.

Binding of NE to HS Moieties of Syn-1 Restricts Anti-elastase Effect of α1-AT

To test if NE binding to HS moieties of Syn-1 restricts anti-elastase effect of α₁-AT, rhSyn-1 with or without prior HS^{ase} I and II treatment was allowed to bind with NE at a concentration commensurate with unopposed NE activity found in sputum samples of patients (2 mM, as determined by active-site titration). Inhibition of NE activity was assessed after standard additions of test inhibitors, α₁-AT or eglin C, into the reaction mixture. The inhibitor effects were compared with those of parallel procedure performed on DTT-pretreated sputum sol samples of the 12 patient recruits. In all cases, inhibition of NE activity increased with inhibitor concentration in the reaction mixture. However, the percent inhibition differed with the anti-elastase used.

With exogenous α₁-AT at 2 m M (1:1 molar equivalence of active-site-titrated NE), 44-46% of NE activity was inhibited in incubations with either rhSyn-1- or DTT-treated sputum samples; even with exogenous α₁-AT at 20 m M (10 times the molar equivalence of active-site-titrated NE), the inhibition could only reach 63-65% in either case. On the contrary, complete inhibition of NE activity was achieved both at 2 and 20 μM α₁-AT for incubations with samples that had been treated with HS^{ase} I & II. Together with the finding that HS cleavage by HS^{ase} I and II treatment of Syn-1 prohibited NE binding, these results suggest that NE binding to HS/Syn-1 limits access and full inhibition of NE by α₁-AT.

Addition of eglin C (an NE-specific tetrapeptide inhibitor) to the reaction mixture, however, resulted in complete inhibition of NE activity in incubations both with rhSyn-1 and with sputum samples, with and without prior HS^{ase} I and II treatment. This contrasted with the differential inhibition seen when Syn-1-bound NE was presented with α₁-AT. The results, therefore, suggest that NE bound to HS moieties of Syn-1 is accessible to inhibition by peptides, but not to large molecular size proteins like the physiological anti-elastase, α₁-AT.

The persistently dominant activity of NE is at the core of the protease-antiprotease hypothesis of airway diseases. In inflammatory secretions of patients with bronchiectasis, the finding of unopposed NE activity reinforces this concept. α₁-AT in molar excesses of unopposed NE has been found in the secretions. Anti-elastase effect of α₁-AT was restricted as a result of NE binding to the HS moiety of Syn-1 in supramolecular complexes of the sputum samples. Reproduction of the phenomenon by incubations of purified NE with rhSyn-1- or DTT-treated sputum complex versus incubations with HS^{ase}-treated counter-parts allowed identification of HS as crucial to NE binding, and thus key to the unopposed NE activity. Although Syn-1 and -4 are both present in the sputum sol samples, and both bear HS moieties that bind NE, the data reinforce the concept that, in the inflamed bronchial environment, the native binding partner of endogenous NE is the HS moiety of Syn-1.

The surface plasmon resonance data show high affinity of NE for HS moieties of rhSyn-1 (K_{d}, 14.17±2.12 nM), similar to that reported for NE binding to the purified Syn-1 ectodomain. Effective competition for NE by heparin was demonstrated, but not by CS. Effectiveness in competing for NE declined in the order of N-desulfated>completely desulfated but re-N-sulfated>>completely desulfated and re-N-acetylated heparins. This corroborates heparin displacement of NE from the supramolecular complex in sputum sol samples, and further suggests that HS moieties that bind NE in the sputum complex are less highly sulfated than heparin. This suggestion is supported by the observations that the HS moieties of DTT-treated sputum complex were susceptible to bacterial HS^{ase} s that specifically act on low-sulfation domains, and that HS cleavage was accompanied by NE release from the sputum complex. It is noteworthy that circulating neutrophils express integral membrane proteoglycans, which bear HS moieties with affinity for NE. These moieties contribute to localization of NE that is released by controlled degranulation during neutrophil migration and extravasation. In the inflamed airways, the HS moieties are likely modified by endoglycosidase activity of heparanase released by activated neutrophils, as demonstrated in human dermal wound fluids. It was inferred, then, that residual HS domains on shed Syns in inflammatory secretions of the patients are of the low-sulfation variety. Thus, not only do the HS moieties of Syn-1 bind NE, as shown in the results, but the data also support that variations in the sulfation domain of HS dictate the binding affinity for NE.

In bronchietatic airways where bacterial colonization and inflammatory response recur, activated neutrophils can resort to a program of nuclear membrane disintegration and granule disassembly within intact cell membranes. Upon membrane fragmentation and cell death, the chromatin and granule contents form “neutrophil extracellular traps” that tether NE. It is expected that there is ready transfer of NE from neutrophil extracellular traps to shed HS/Syn-1 in the inflammatory environment, given that the affinity of NE-polynucleotide (K_{d}, 4 m M to 21 nM) is lower than that of NE-HS/Syn-1). Importantly, NE bound to HS/Syn-1 in the sputum complex remains proteolytically active. The data reinforce the concept that NE activity persists despite molar excesses of α₁-AT.

The new finding of affinity of α₁-AT for the Syn-1 core protein suggests that, in the inflamed bronchial environment, variously deglycanated forms of shed Syn-1 bind α₁-AT, and restrict it from exerting its anti-elastase effect. Even in situations in which the HS moiety was experimentally cleaved by bacterial HS^{ase} s to allow for NE inhibition by α₁-AT, the percent inhibition of NE activity corresponded to standard additions of exogenous α₁-AT. A shift in the response profile to the left would have indicated that endogenous α₁-AT played a part in the inhibition of NE in incubations in which sputum sol samples were included; however, the shift was not observable. The data therefore suggest a new function of the Syn-1 core protein in binding of α₁-AT and thus restricting α₁-AT from inhibiting NE. This restriction is compounded by NE binding to the HS moiety of shed Syn-1, as in the sputum sol samples of patients with bronchiectasis.

TABLE 3
LANGMUIR 1:1 MODEL FOR BINDING OF NEUTROPHIL
ELASTASE TO IMMOBILIZED HEPARAN SULFATES

| Binding Constants | Mean ± SD |
|---|---|
| kₐ × 10⁴ M⁻¹s⁻¹ | 3.31 ± 0.14 |
| k_{d} × 10⁻⁴ s⁻¹ | 4.69 ± 0.21 |
| K_{d}, nM | 14.17 ± 2.12 |

Definition of abbreviations: kₐ, yield rate constant for association; k_{d}, yield rate constant for dissociation; K_{d}, equilibrium dissociation constant.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A pharmaceutical dosage unit for administration to the pulmonary tract or mucosa comprising a pharmaceutically acceptable agent in an amount effective to decrease neutrophil elastase activity in a subject in need thereof, to reduce, prevent, or inhibit one or more biochemical measures or symptoms associated with a respiratory disease or disorder.

2. The pharmaceutical dosage unit of claim 1 further comprising a pharmaceutically acceptable carrier for pulmonary or nasal administration.

3. The pharmaceutical dosage unit of claim 1 wherein the agent disrupts or prevents binding of neutrophil elastase to syndecan-1.

4. The pharmaceutical dosage unit of claim 1 wherein the agent is a glycosaminoglycan.

5. The pharmaceutical dosage unit of claim 4 wherein the glycosaminoglycan is selected from the group consisting of heparin or heparin derived fragments, and combinations thereof that do not possess anti-coagulant activity.

6. The pharmaceutical dosage unit of claim 5 wherein the heparin derived fragment is a tetra-, hexa- or octasaccharide.

7. The pharmaceutical dosage unit of claim 5 wherein the heparin or heparin-derived fragment, and combinations thereof contains between 1 and 10 glucuronic acid residues.

8. The pharmaceutical dosage unit of claim 2 in the form of particles, aerosol, or spray.

9. The pharmaceutical dosage unit of claim 2 wherein the carrier is chitosan or a chitosan derivative.

10. The pharmaceutical dosage unit of claim 9 wherein the chitosan is in the form of or a coating on a microsphere between 1 μm and 10 μm in diameter.

11. The pharmaceutical dosage unit of claim 1 further comprising a second therapeutic agent selected from the group consisting of protease inhibitors, anti-elastases, anti-inflammatories, mucolytics, antibiotics, antivirals, bronchodilators, β2 agonists, anticholinergics, theophylline, and corticosteroids.

12. A method of treating a respiratory disease or disorder comprising administering to a subject in need thereof the dosage unit of claim 1.

13. The method of claim 12, wherein the agent is administered in an amount effective to reduce, treat, inhibit, or alleviate one or more symptom of chronic respiratory inflammation.

14. The method of claim 12, wherein the agent is administered to an individual with chronic respiratory inflammation.

15. The method of claim 14 wherein the chronic respiratory inflammation is bronchiectasis, emphysema, or chronic obstructive pulmonary disease.

16. The method of claim 13 wherein the symptoms are prolonged or abnormal inflammation, permanently dilated bronchi, airflow limitation, chronic cough, sputum production, hemoptysis, dyspnea, air trapping, wheezing, chest pain or recurrent lung infection.
